(19) United States
(12) Patent Application Publication
(10) Pub. No.: US 2025/0193549 A1
(43) Pub. Date: Jun. 12, 2025

(54) TIME DELAY SUMMATION FOR MOVING OBJECTS

(71) Applicant: VAREX IMAGING SWEDEN AB, Danderyd (SE)

(72) Inventors: Christer Ullberg, Sollentuna (SE); Alexander Stewart, San Francisco, CA (US); Mattias Urech, Nacka (SE); Atukem Nabina, London (GB); Tuomas Pantsar, Espoo (FI)

(21) Appl. No.: 18/917,376

(22) Filed: Oct. 16, 2024

(30) Foreign Application Priority Data
Dec. 12, 2023 (SE) 2351417-7

(51) Int. Cl. H04N 25/768; H04N 25/30; H04N 25/46

(52) U.S. Cl. CPC H04N 25/768; H04N 25/30; H04N 25/46

(57) ABSTRACT

A device for generating an X-ray image is provided. The device obtains first and second sets (350-(t−1), 350-t) of pixel frame data from readouts of sensor pixels (SP1-SPN) at first and second times (t−1, t). The sensor pixels are spatially offset from one another by a first distance (dy) in a sensor scanning direction (y). Between the first and second times, an object point-projection (122a, 122b) on the sensor has had time to move a distance (h) greater than the first distance. A first data element (Li(t−1)) of the first set is combined (236) with a second data element (Li(t)) of the second set, wherein the first and second data elements are associated with different sensor pixels, to generate (part of) the image 400. A corresponding X-ray detector, imaging system and method are also provided.

### CROSS-REFERENCE TO RELATED APPLICATION(S)

This application claims priority to Swedish Application No. 2351417-7, filed 12 Dec. 2023, and entitled “TIME DELAY SUMMATION FOR MOVING OBJECTS,” the disclosure of which is incorporated herein by reference in its entirety.

### BACKGROUND

In many situations, it may be desirable to create still images of one or more objects that are moving relative to an imaging device, such as an X-ray detector and similar. Use-cases include e.g. airport security, inspection of food or other products, medical imaging, toll/customs operations, various recycling scenarios, and inspection of electronics equipment.

So-called time delay integration (TDI) or time delay summation (TDS, in case of e.g. photon-counting detectors) relies on adjusting a readout frequency of the detector such that it matches the movement of the object relative to the detector. In a detector including a sensor with multiple lines of sensor pixels, a time between consecutive readouts of all sensor pixel values may be adjusted such that a first line of sensor pixels sees a point of the object at a first readout time instance, an adjacent second line of sensor pixels sees the same point of the object at a consecutive second readout time, and so on. By scanning the object in this way, and by combining data read out from the various lines of sensor pixels at different time instances, a sharp still image of the object may be obtained even though the object is moving relative to the detector. The time between consecutive readouts of all sensor pixel values may be defined in terms of a “readout frequency”.

### BRIEF DESCRIPTION OF THE DRAWINGS

FIG. 1 schematically illustrates an exemplary imaging system, as well as a device for generating X-ray images of a moving object, according to the present disclosure.

FIG. 2 schematically illustrates an exemplary X-ray detector and device according to the present disclosure.

FIGS. 3A to 3G schematically illustrate various exemplary sensor data readout scenarios according to the present disclosure.

FIGS. 4A to 4D schematically illustrate various exemplary pixel line data accumulation scenarios according to the present disclosure.

FIGS. 5A and 5B schematically illustrate exemplary post-processing modules according to the present disclosure.

FIGS. 6A to 6C schematically illustrate various exemplary post-processing modules according to the present disclosure.

FIG. 7 schematically illustrates a flowchart of exemplary methods for generating X-ray images of a moving object according to the present disclosure.

FIG. 8 schematically illustrates exemplary devices according to the present disclosure in terms of functional modules.

FIG. 9 schematically illustrates post-readout processing performed by an exemplary device according to the present disclosure.

### DETAILED DESCRIPTION

Readout from a sensor does not happen instantly, but takes a certain amount of time during at least part of which the detector is blocked from detecting new photons arriving from the object. For example, the detector may be blocked from detecting new photons at least during reset of the sensor pixel values, but may in some situations also be blocked during the actual readout of the sensor pixel values itself. In any case, the duration of the time during which the detector is blocked from detecting new photons usually does not depend on the readout frequency, but remains constant independently of whether the detector/sensor is read out more or less frequently. As a consequence, a ratio of such “blocked time” to time used to detect photons thus increases as the readout frequency increases, as a larger share of the overall time is spent on e.g. resetting (and possible also on reading out) sensor pixel values instead of on detecting photons.

A problem with contemporary TDI/TDS solutions is thus that in order to image an object which moves more quickly relative to the sensor, the readout frequency must be increased to still match the movement of the object, with the above-indicated disadvantages. In particular, as the proportion of blocked time increase, the overall sensitivity of the detector goes down as fewer photons are detected.

Yet another problem with contemporary TDI/TDS solutions is that the sheer amount of data that needs to be read out from the sensor and then processed in order to create a still image of the moving object also increases as the readout frequency increases, which may lead to a need for additional computational resources or exceeding a capability of the computational resources at hand. Binning of one or more sensor lines/pixels after readout from the sensor and before further processing may help to reduce the amount of data required to processed, but still does not overcome the reading time issue described above.

For example, if a sensor with 60 lines and a line pitch of 0.1 millimeters (mm) is supposed to image an object moving with a speed such that the projection of an object point/feature on an area of the sensor moves with 1000 millimeters per second (mm/s), the readout frequency should match 10000 Hz (i.e. 1000 mm/s divided by 0.1 mm). For higher-resolution images, it may be desirable to use even smaller pixels than that, leading to an even further increase in required readout frequency.

For all of the above-indicated reasons, generating still images of more quickly moving objects may thus be challenging. The present disclosure aims at improving on the current situation by providing an improved device for generating an X-ray image, a corresponding method, a detector including such a device, as well as an imaging system including such a device and/or detector. The envisaged device and other entities are well suitable also for processing data from multiple sensors in parallel, such as in a detector including a plurality of (multiline/-pixel) sensors.

These improved contributions over contemporary technology will now be described in more detail in what follows. When referring to the accompanying drawings and the figures thereof, same or similar reference numerals will be used to denote the same or similar structural/logical features.

As generally used herein, the term “readout” or “reading out of sensor pixel values” refers to processing of (substantially) all of a plurality of physical sensor pixels, despite whether the processing takes place inside e.g. an application-specific integrated circuit (ASIC), on a field-programmable gate-array (FPGA), or on e.g. a central processing unit (CPU), microcontroller unit (CPU), graphics processing unit (GPU), and similar.

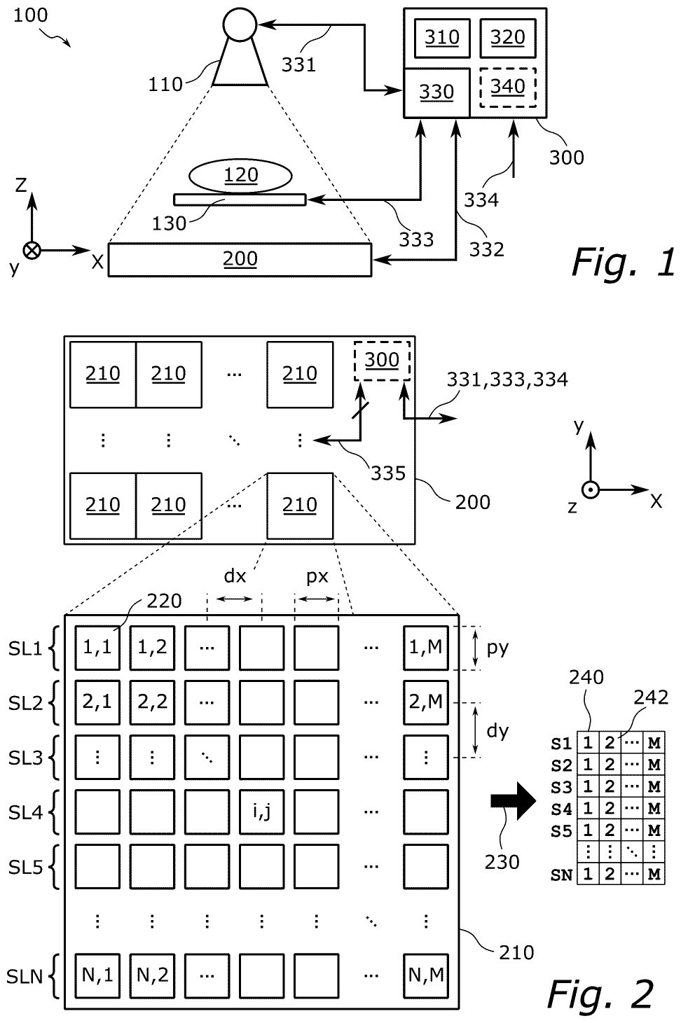

FIG. 1 schematically illustrates an example of an imaging system 100. The system 100 includes a radiation source 110 and a radiation detector 200. The source 110 may be an X-ray tube or similar for emitting X-rays through an object 120 that is to be imaged, and the detector 200 may correspondingly be an X-ray detector configured to detect such X-rays after they have passed through the object 120. The detector 200 may operate by detecting the incoming radiation (e.g. X-rays) and converting such incoming radiation into electric signals that may be further processed in order to generate a spatially resolved projection image of the object 120.

The system 100 further includes a device 300 that may be configured to perform various tasks such as one or more of controlling the source 110, reading data from the detector 200, processing the data read out from the detector 200 as part of generating an image (such as an X-ray image) of the object 120, controlling a motion apparatus 130, and similar.

The device 300 includes processing circuitry 310. The device 300 may also include a memory 320 with which the processing circuitry 310 may communicate in order to read/store data from/in the memory 320. The device 300 may also include a communications interface 330 with which e.g. the processing circuitry 310 (and e.g. the memory 320) may communicate with the source 110 (by exchanging data 331), with the detector 200 (by exchanging data 332), and e.g. with the motion apparatus 130 (by exchanging data 333). Such communication may be wired and rely on electric and/or optical signals transmitted through one or more suitable wires, and/or be wireless and rely on the exchange of electromagnetic signals. The device 300 may optionally include one or more other entities (here illustrated by the dashed box 340) to perform other functions than those listed above. The device 300 may optionally also exchange other data 334 with one or more external devices, such as a user interface, a user terminal, a server, and similar. As will be elaborated on later herein, the device 300 may be divided into, or include, multiple functional units each configured to perform a specific task. Each such unit may include its own processing circuitry (and e.g. memory), or two or more such units may share a same processing circuitry (and e.g. memory), if being e.g. logical units implemented with software only, and similar. As envisaged herein, the memory 320 may store instructions which, when read and executed by the processing circuitry 310, may cause the device 300 to perform various functions as described herein. Some such functions may be performed by the device 300 itself, while other such functions may be performed by some other entity but commanded by the device 300.

The processing circuitry 310 is provided using any combination of one or more of a suitable central processing unit (CPU), multiprocessor, microcontroller, digital signal processor (DSP), etc., capable of executing the instructions stored in the memory 320. The processing circuitry 310 may further be provided as part of at least one application specific integrated circuit (ASIC), or field-programmable gate array (FPGA). The memory 320 may be provided as a random-access memory (RAM), a read-only memory (ROM), an erasable programmable read-only memory (EPROM), or an electrically erasable programmable read- only memory (EEPROM) and/or as a non-volatile storage medium of a device in an external memory such as a USB (Universal Serial Bus) memory or a Flash memory, such as a compact Flash memory. The memory 320 may also include persistent storage, which, for example, can be any single or combination of magnetic memory, optical memory, solid state memory or even remotely mounted memory.

As envisaged herein, the system 100 can be used to generate images of moving objects (such as object 120), which will herein be referred to as the detector 200 “scanning” or “imaging” the object 120. As used herein, “moving” implies “relative movement”. Phrased differently, it is not necessarily such that the detector 200 is fixed and the object 120 moves. To the contrary, it is envisaged that relative movement may instead be caused by the object 120 being fixed and the detector 200 moving relative the object 120, or by both the object 120 and the detector 200 moving. One or both of the object 120 and detector 200 may move in for example the y-direction as illustrated in FIG. 1. For this purpose, a motion apparatus 130 may be provided in order to provide such relative motion of the detector 200 and object 120. For example, the motion apparatus 130 may be a conveyor belt configured to move the object 120 in the y-direction. In other envisaged examples, the motion apparatus 130 may instead be configured to move the detector 200. When moving the detector, it is implied that the source 110 also moves along with the detector 200, and/or that an X-ray beam emitted by the source 110 is wide enough such that the detector 200 may receive sufficient radiation even when the detector 200 is moving and the source 110 kept fixed, and vice versa. Phrased differently, it is envisaged herein that radiation emitted by the source 110 may be received by the detector 200 independently of where the detector 200 is currently located relative the object 120. Other configurations are of course also possible. It should be noted that what matters the most is not the movement of the whole object relative the detector 200, but rather a movement of a projection of a point/feature of the object 120 on a surface of a sensor of the detector 200. This movement of the projection may not necessarily correspond to the movement of the object itself, as how the projection moves across the sensor will likely depend on e.g. a distance between the object 120 (point/feature) and the sensor, etc.

The detector 200 may for example mainly extend in the x-and y-direction, and the source 110 may be arranged at a distance from the detector 200 in the z-direction, as also illustrated in FIG. 1. As envisaged herein, the system 100 may be used to perform one or many of computed tomography (CT) scanning of the object 120, X-ray scanning of the object 120, and similar. The detector 200 is often not large enough to capture a whole of the object 120 at once, in a single exposure/readout, but needs to capture one or more subsequent images of the object 120 using scanning. Other examples of how the system 100 may be used to generate scanning images of the object 120 are of course also possible, and the few examples listed herein does not provide an exhaustive list of all such possible examples.

As envisaged herein, the device 300 may also be an integrated part of the detector 200. This may include all of the device 300 as described so far, or e.g. at least part of the device 300 to perform detector-specific tasks.

FIG. 2 schematically illustrates an exemplary detector 200 in more detail, as seen from above. More specifically, the detector 200 includes one or more multiline sensors 210, such as one or more multiline X-ray sensors. If more than one sensor 210 is included as part of the detector 200, the sensors 210 may be arranged in a line, or in multiple lines in order to form a grid pattern such as shown in FIG. 2, or in any other pattern (including also 3D patterns, wherein two nearby sensors are separated also in the z-direction), and similar. The detector 200 may further include the device 300, which may be configured to communicate with the one or more sensors 210 by exchanging data 335. As generally used herein, “exchanging data” may correspond to e.g. sending/generating or receiving/reading an electric signal, an optical signal, an electromagnetic signal, or similar, and/or by reading/writing from/to a memory, such as e.g. the memory 320. If included as part of the detector 200, the device 300 may optionally be configured to communicate with one or more external devices, for example by exchanging data 331 with the source 110, data 333 with the motion apparatus, and e.g. additional data 334 with one or more additional, external devices.

Shown in FIG. 2 is also an example sensor 210 in more detail. The sensor 210 is “multiline” as it includes a plurality of lines SL1, SL2, . . . , SLN of sensor pixels 220, where N is an integer indicating a total number of such lines of sensor pixels 220. The pixels 220 are thus, in this example, arranged in a rectangular lattice pattern. A spacing/pitch between neighboring pixels 220 in the x-direction is denoted dx, and a size (e.g. width) of each individual pixel 220 in the x-direction is denoted px. Likewise, a spacing/pitch between neighboring pixels 220 in the y-direction is denoted dy, and a size (e.g. height) of each individual pixel 220 in the y-direction is denoted py. The spacing/pitch dy between neighboring pixels 220 in the y-direction may in this example also be referred to as a “sensor line pitch” or similar. In total, the sensor 210 has N lines of sensor pixels, wherein each line includes M sensor pixels, where M is also an integer. As an example, each pixel 220 may be addressed using two indices i, j wherein i denotes the line (row) of the pixel 220 and j denotes the column of the pixel 220. In other envisaged embodiments of a sensor, the sensor pixels 220 may be arranged in other patterns than the regular/rectangular pattern shown in FIG. 2. For example, instead of in straight lines, the sensor pixels 220 may be arranged in curved lines (i.e. arcs). Likewise, the sensor pixels 220 do not necessarily all lie in a same plane, but may be arranged such that e.g. two neighboring sensor pixels 220 are separated from each other in the z-direction, and similar. Sensor pixels 220 may also, in some examples, be arranged in more complex patterns, e.g. in accordance with a triangular lattice, a honeycomb lattice, a hexagonal lattice, etc. The spacings/pitches px and py may be equal or different. In some examples, it is envisaged that the spacing/pitch between neighboring sensor pixels 220 considered to lie on/in a same “line” may also be different between different sensor pixels 220, etc. Even more generally, it may be assumed that the spacings/pitches px and py may be different for any two adjacent/neighboring sensor pixels 220. For example, if the object 120 that is to be imaged is curved, spherical, etc., it may be beneficial to arrange the sensor pixels 220 such that the sensor pixels 220 are arranged closer together towards a center of the sensor 210 than towards an outer edge of the sensor 210, or vice versa.

The device 300 is such that it may read out pixel data from each of the pixels 220 of each sensor 210, e.g. as part of data 335. Such readout may be performed by the device 300 having individual communication channels to all pixels 220, or by using one or more multiplexers configured for such purposes. Generally herein, the detector may utilize either direct or indirect conversion of impinging X-ray photons to electrons. Indirect conversion detectors may utilize a scintillator such as one based on gadolinium (e.g. GOS, Gadox, or Gd₂O₂S) or cesium iodine (CsI), to first convert X-ray photons to visible light, convert the visible light to electrons using e.g. (silicon) photodiodes, charge-coupled devices (CCDs), or complementary metal-oxide semiconductor (CMOS) devices, and then read out the electrons using e.g. the CCD/CMOS itself or a thin-film transistor (TFT) arrangement. Direct conversion detectors may skip the “X-ray to visible light” step by using a material in which impinging X-ray photons are directly converted to one or more electron-hole pairs, and wherein the electrons are read out using e.g. a TFT or thin-film diode (TFD) array, or a CMOS. The direct conversion may be achieved using e.g. amorphous selenium (a-Se). Other envisaged types of direct conversion detectors may include so-called photon-counting detectors, and in particular those using cadmium telluride (CdTe) or cadmium zinc telluride (CdZnTe or CZT) for the direct conversion element (and such materials may also be used in non-photon counting detectors). Here, the operating principle relies on applying an electric field across the direct conversion element, such that one or more electron-hole pairs created in the material (i.e. in response to absorption of energy provided by an impinging X-ray photon) may be split and transferred to respective sides of the element. An electrode placed on e.g. one of the sides can then be used to output a signal caused by the movement of charge in the electric field, and the signal can be measured using a readout circuit (such as an application-specific integrated circuit, ASIC). Generally, it is assumed that the number of generated electron-hole pairs is proportional to the energy deposited by the X-ray photons. For CdTe/CZT material with low hole mobility, the electrode can be arranged to collect the electrons. The readout circuit may e.g. include one or more comparators each comparing the signal from the electrode with a particular threshold. Each time a comparator detects that the signal exceeds its particular threshold, the comparator may output a signal to a counter which in turn increases its count with one. By using multiple comparators each having their own and unique thresholds, such a readout circuit may be capable of also counting photons at different energies. The readout circuit may thus keep track of how many of the incoming photons that had an energy exceeding a first energy threshold, how many of the incoming photons that had an energy that also exceeded a second energy threshold, and so on. The exact energy thresholds may be user-configurable, and defined by properly tuning the signals to which the one or more comparators compare their respective input signal from the electrode. A photon-counting detector may be configured to operate in different so-called counting modes, such as e.g. a non-paralyzable mode and a paralyzable mode. Other logic and/or physical adaptations may also be provided to handle e.g. charge sharing and similar. For photon-counting detectors, it is of course envisaged that there are multiple electrodes provided, and that each electrode thus corresponds to a single sensor pixel. Phrased differently, the photon-counting detector is capable of counting a number of photons arriving in an area corresponding to each sensor pixel, and possibly also bin such counts into multiple bins each corresponding to a particular photon energy level. If considering a single photon energy level (such as a lowest photon energy level), the detector may be referred to as operating in a single-energy mode. If considering two or more photon energy levels, the detector may be referred to as operating in a multi-energy mode (such as a dual-energy mode, triple-energy mode, or similar).

The readout of each sensor 210 of the detector 200 is performed in so-called readout intervals. Using a photon-counting detector as an example, at the beginning of a readout interval, the one or more counters associated with each of the sensor pixels 220 of the sensor 210 are reset (to e.g. zero). As photons arrive, the one or more counters will be increased as described above. At the end of the readout interval, the current counts of the one or more counters of each sensor pixel are read out (referred to as a readout operation 230 of the sensor 210), and the counters for all sensor pixels are then once again reset such that a new readout interval may be subsequently started. As used herein, a readout interval may be bounded/defined by a pair of readout time instances.

Generally herein, for each sensor 210, the readout operation 230 thus provides sensor readout data 240 indicative of at least how many photons that have impinged on or at each sensor pixel 220 of the sensor 210, possibly also for different energy levels/bins as described above, during the corresponding readout interval. For a photon-counting detector, this data may include actual counts of photons, while other types of detectors may provide data indicative of e.g. an integral of received photon energy over time, or similar. In any way, it is envisaged that the sensor readout data 240 may be structured as data for a plurality of sensor pixels, for example as a number of sensor readout data lines S1, S2, . . . , SN (where N is the integer number of total lines of sensor pixels). For each such data line, there is provided data for a plurality of sensor pixels 1, 2, . . . , M (where M is the integer number of sensor pixels per line). In other envisaged examples, the sensor readout data 240 may e.g. be structured as a number of sensor pixel readout data elements, where each element provides data for a single sensor pixel 220. In any way, for e.g. a photon-counting detector, each cell 242 of the sensor readout data may thus correspond to a count of photons for a particular sensor pixel 220 (that may or may not belong to a particular line of sensor pixels). For a multi-energy detector, there may be several sets of sensor readout data 240, each set corresponding to a particular energy level, and similar. The sensor readout data 240 may also be referred to as an “exposure frame” of the sensor 210.

A first main idea underlying the present disclosure is to reduce the readout frequency on purpose, thus reducing the time spent on reading out the sensor (including at least blocked time) compared to the time available for detecting photons. In particular, it is envisaged to reduce the readout frequency such that between two consecutive readouts, such as between first and second readout time instances, the object 120 imaged by the sensor 210 has sufficient time to move a distance relative the sensor 210 causing motion blur of the object 120. More in particular, it is envisaged to adjust/reduce the readout frequency such that for a particular point (or feature) of the object 120, the projection of that point on a surface of the sensor 210 moves a distance larger than a spacing/pitch of the sensor pixels 220 in the scanning direction y of the sensor. For example, the object point-projection can move a distance spanned by more than one (i.e. K>1, such as e.g. K≥2 if K is an integer) consecutive lines of sensor pixels 220 (or e.g. by just more than one sensor pixel 220). For example, if the point of the object 120 is projected at a line SLᵢ of sensor pixels 220 at a first readout time instance, the point of the object 120 will be projected at a line SL_{i′} at a next, consecutive readout time instance, where i′≥i+K (where K>1, such as e.g. K≥2). Reducing the readout frequency in this way reduces the amount of data that needs to be transferred between the sensor 210 and e.g. the device 300 over time, and thereby helps to reduce the ratio of “blocked time” to overall time and at least partially resolves the above-identified problem with contemporary technology. For notice, it should be noted that in contemporary TDS/TDI solutions, the readout frequency is higher and such that during two consecutive readout time instances, the projection of the object point has the time to move a single line of sensor pixels 220 (e.g. a distance equal to a spacing/pitch of the sensor pixels 220 in the scanning direction y), i.e. such that (using the above example) i′=i+1.

As mentioned already, it should be noted that the concept underlying the present disclosure is also applicable even if there are no well-defined “lines”/“arcs”/“strings” of sensor pixels as illustrated in FIG. 2. For example, the sensor 210 may be one-dimensional and have a single line of sensor pixels 220 oriented along the scanning direction of the sensor 210, such as e.g. along the y-direction as illustrated in FIG. 2. One may then say that the readout frequency is to be adjusted such that between two consecutive readouts of all the sensor pixel values, the projection of the object has time to move K consecutive sensor pixels (instead of two or more consecutive lines of sensor pixels) in the scanning direction, and/or e.g. a distance larger than a spacing/pitch of the sensor pixels in the scanning direction. Phrased differently, it can be assumed that the sensor pixels are each spatially offset (to one another) by a first distance in the scanning direction, and the readout frequency is adjusted such that between first and second readout time instance, the object point-projection on the sensor has time to move a second distance larger than the first distance in the scanning direction y. Likewise, even if there are well-defined lines each including more than one sensor pixel 220, the present concept may still be discussed in terms of such a one-dimensional sensor.

As the readout frequency is, in accordance with the present disclosure, adjusted such that the projection of an object point on the sensor 210 has time to move a distance larger than that spanned by more than one line of sensor pixels 220 (or e.g. more than one sensor pixel 220) between two consecutive readouts, photons originating from this object point will be registered/detected by sensor pixels 220 belonging to multiple lines of sensor pixels 220 (or by multiple sensor pixels 220 along the scanning direction of the sensor 210) during a same readout interval. As a consequence, there will be motion blur introduced when attempting to image the object 120 as it moves relative to the sensor 210. In return, less time will be spent on blocked time/readout of the sensor, thus enabling an increase in sensitivity of the sensor 210 and associated detector 200.

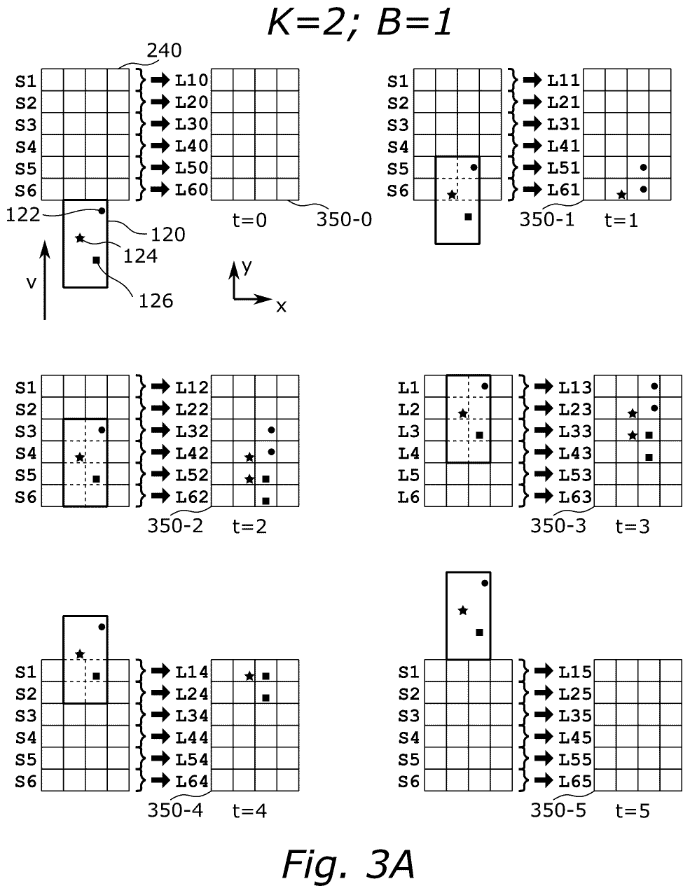

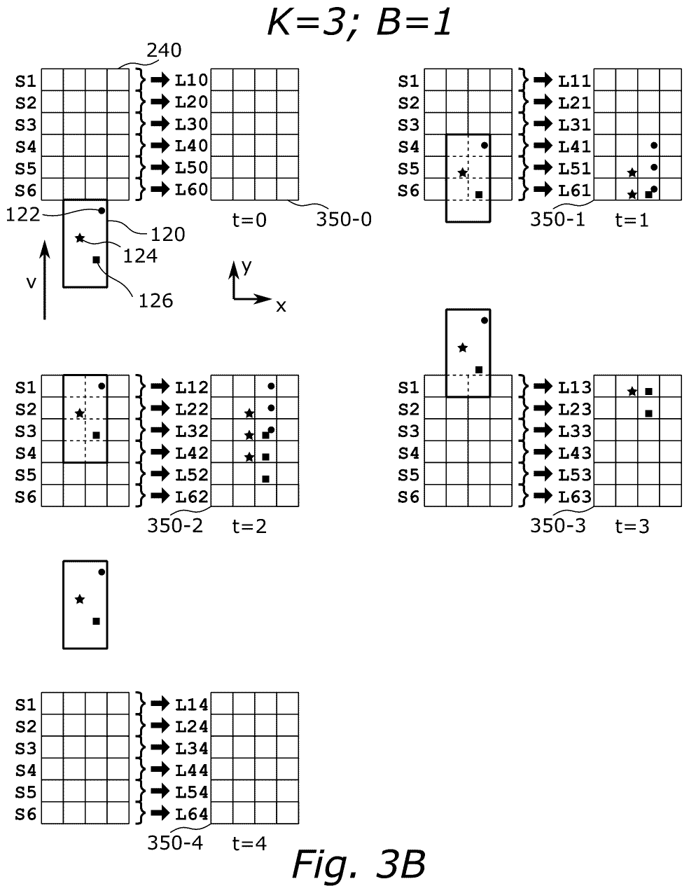

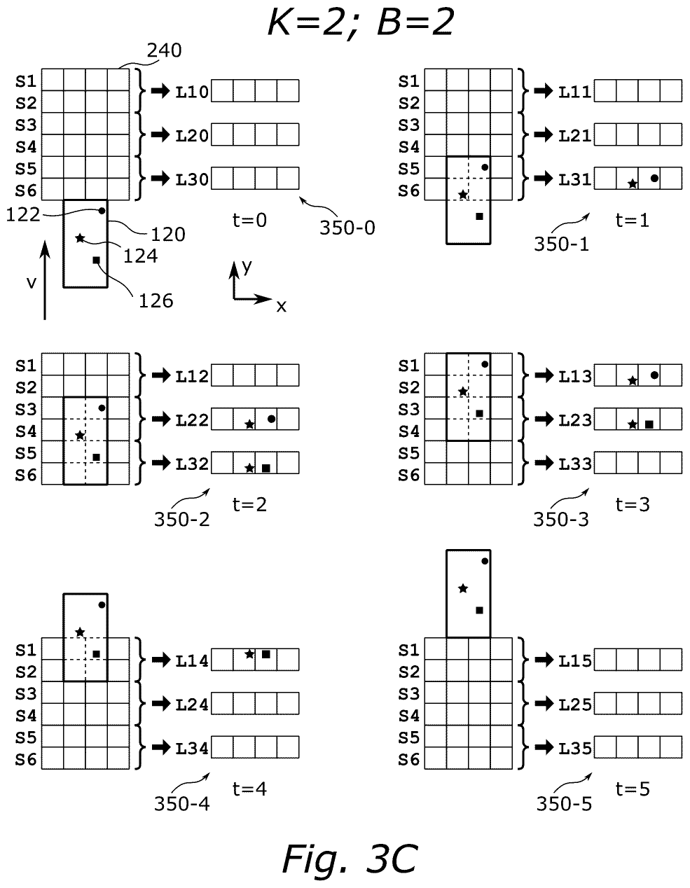

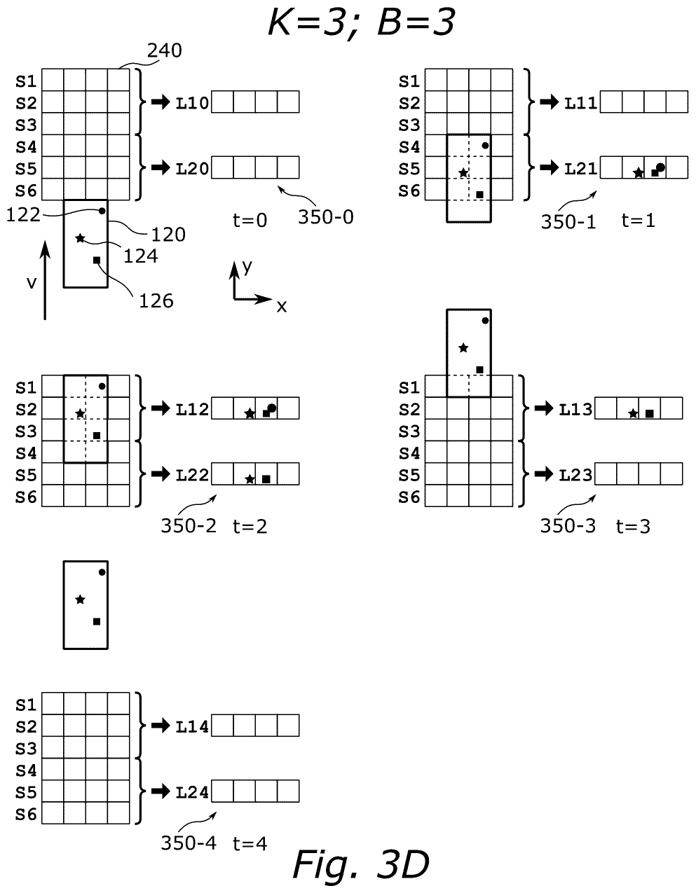

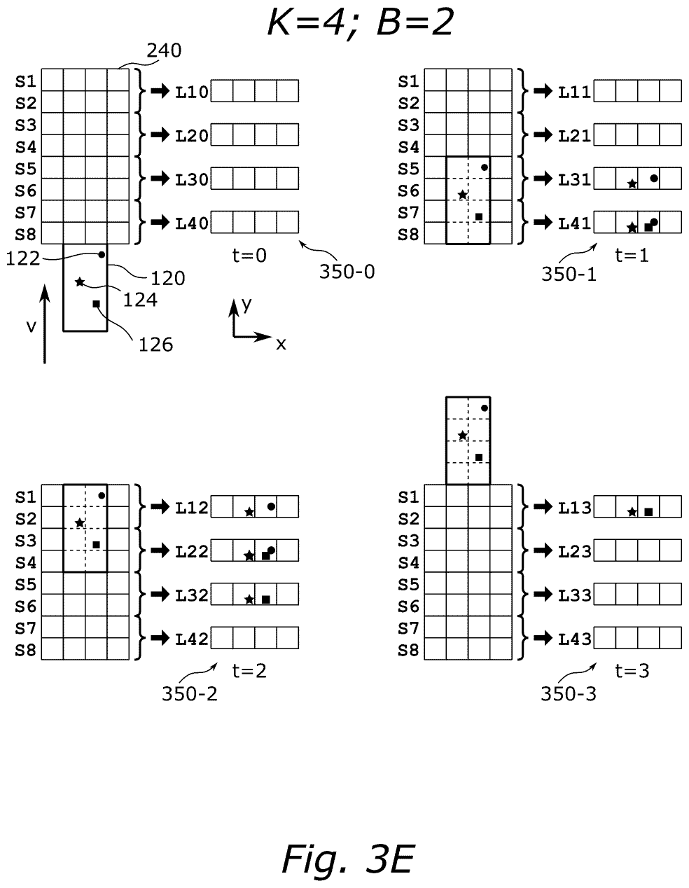

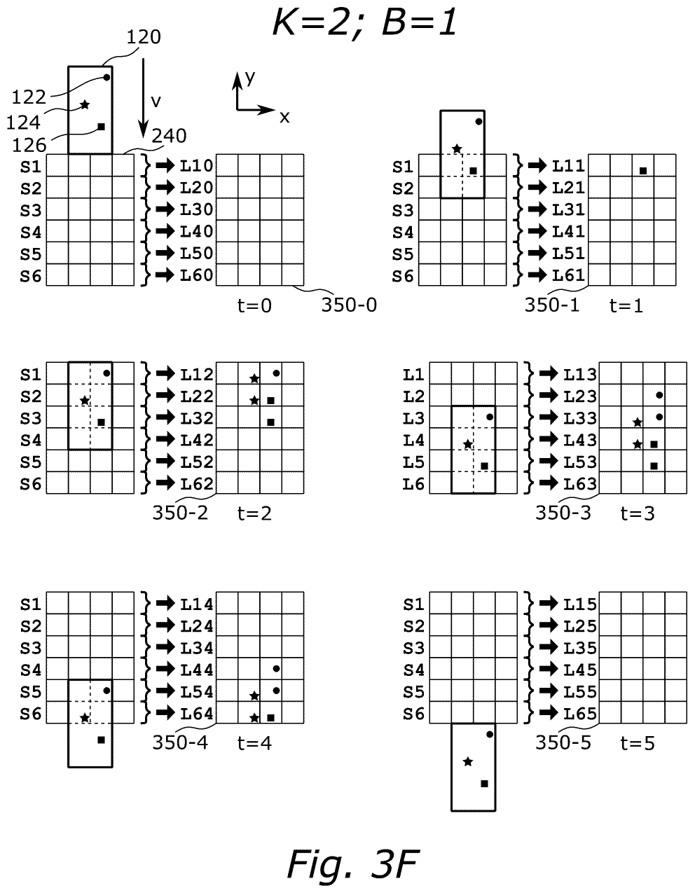

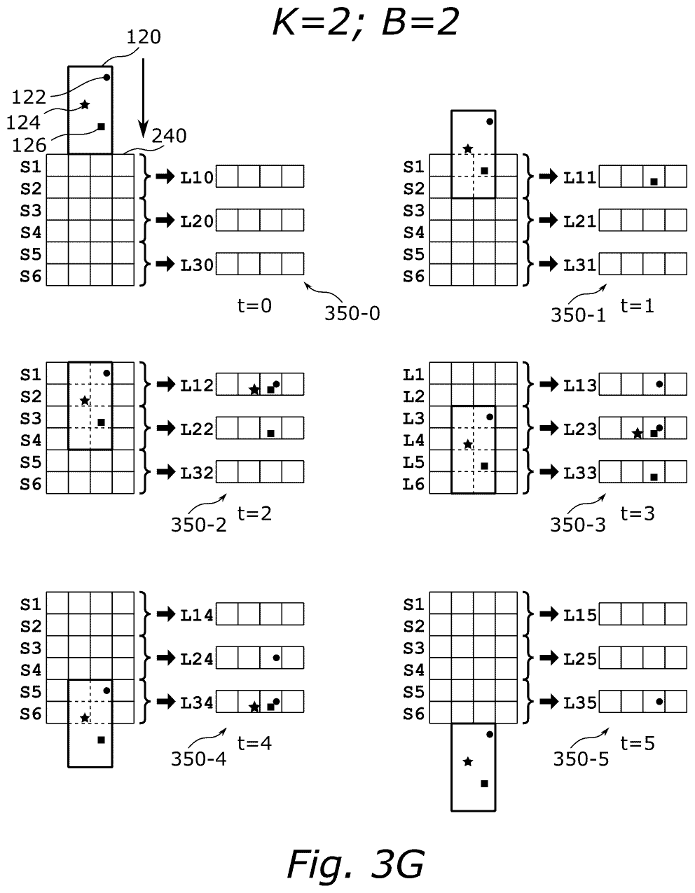

Various examples of how the readout frequency can be reduced will now be described in more detail with reference also to FIGS. 3A-3G. In these Figures, it is assumed that the object point-projection on the sensor moves an integer number K≥1 of lines of sensor pixels 220, i.e. such that the distance moved by the object point-projection is K times the first distance (pitch/spacing) between adjacent lines of sensor pixels 220 in the scanning direction y. Some of these examples will also illustrate the concept of binning two or more lines of sensor pixels together to further reduce the amounts of data having to be processed, as is also envisaged herein. Here, it is assumed that an integer number B lines of sensor pixels 220 are binned/grouped together, where B=1 is used to indicate that no such binning/grouping is performed.

FIG. 3A schematically illustrates one example of the proposed concept. For illustrative purposes, the sensor 210 is here assumed to have six lines SL1-SL6 of sensor pixels 220, with each line including four sensor pixels 220. The corresponding sensor readout data 240 may thus be structured into an array of six lines S1-S6 each having four (pixel) data elements 242. For this particular example, it may be assumed that the detector 200 is a photon-counting detector operating in single-energy mode, and that each element 242 in the data 240 thus corresponds to a single count of all impinging photons having exceeded a lowest photon energy threshold for the corresponding sensor pixel 220 during the readout interval ending with the current readout. In this example, the object 120 moves K=2 lines of sensor pixels 220 during two consecutive sensor readouts, and there is no binning of lines of sensor pixels 220 (i.e. B=1).

For illustrative purposes, the object 120 is in this example considered to have three object points 122, 124 and 126 of particular interest. The exact shape/contour of the object is considered irrelevant, and the movement of the points 122, 124 and 126 (as projected on the sensor 210) will be discussed. The projections of the points 122, 124 and 126 are considered to move with a fixed speed |ν| in the direction indicated in by the arrow/vector ν in FIG. 3A. Here, it is assumed that the direction ν is transverse to the x-direction and parallel to the y-direction. Each line of sensor pixels 220 extend along the x-direction, and the y-direction is considered a scanning direction of the sensor 210. In this example, the object moves from the bottom of the sensor 210 (line SL6) towards the top of the sensor (line SL1), which may also be referred to as a “forward scanning direction” of the sensor.

At a first readout time instance t=0, the object 120 is still outside the view of the sensor 210, and no photons are thus counted by the various sensor pixels 220 (at least no photons associated with/originating from any of the object points 122, 124 and 126). The sensor readout data 240 is thus empty, wherein “empty” is taken to mean that there are no counts therein of any photons originating from the object points 122, 124 and 126. Generally herein, it is assumed that the sensor readout data 240 may be modified before being processed, which is represented by the sensor readout data 240 obtained at t=0 being transformed into a set of pixel frame data 350-0. The set of pixel frame data 350-0 includes data for a plurality of data elements, here grouped into a plurality of pixel lines L1t, L2t, . . . , where t is an integer indicating the current time instance. Thus, for the time instance t=0, the set of pixel frame data 350-0 includes a plurality of data elements for pixel lines L10, L20, . . . . In this particular example, there is no binning (or other pre-processing) of the sensor readout data 240, and the number of pixel lines in the set of pixel frame data 350-0 is thus the same as the number of lines SL1, SL2, . . . of sensor pixels 220, and the number of data elements in the set of pixel frame data 350-0 is the same as the number of sensor pixels 220. In this case, there is thus six pixel lines L10-L60. It should however be noted that pre-processing of the sensor readout data 240 may also reduce the total number of pixel lines S1, S2, . . . , e.g. by skipping data originating from the first and last lines SL1 and SLN of sensor pixels 220, by performing binning of the lines of sensor pixels 220, and similar, as will be exemplified in more detail later herein. As mentioned earlier herein, binning may e.g. include combining every B lines of sensor pixels 220 into a single output line in the pixel frame data 350, such that e.g. lines SL1, . . . , SLB of sensor pixels 220 are combined into a pixel line L1t, lines SL(B+1), . . . , SL(2B) are combined into a pixel line L2t, and so on. In this particular example, there is no such binning, and B=1.

At a next consecutive readout time instance t=1, the respective projection of each of the object points 122, 124 and 126 on the sensor 210 has now had time to move a distance corresponding to two lines of sensor pixels 220 (as K=2), meaning that the readout frequency has been adjusted accordingly. Compared with conventional TDI/TDS solution, the readout frequency is considered to be one half of the conventional TDI/TDS readout frequency. Herein, the readout frequency and the speed of movement of the object point projections may be characterized by the number K, indicating how many lines of sensor pixels 220 that spans the projections movement during two consecutive readout time instances. In this particular example, K=2 as the projections each move a distance corresponding (spanned by) to two lines of sensor pixels 220 between consecutive readout time instances. As the projection of e.g. the object point 122 visits both the lines SL6 and SL5 of sensor pixels 220 during this readout interval, photons originating from the object point 122 will be detected/counted by sensor pixels 220 in both of these lines. This is illustrated in the corresponding set of pixel frame data 350-1 obtained for this time instance, by the symbol (circle) of the object point 122 appearing both in pixel lines L61 and L51. The object point 124 has had time to visit the line SL6 so far, illustrated by the symbol (star) of the object point 124 being present in the pixel line L61 of the set of pixel frame data 350-1.

As the projections of the object points 122, 124 and 126 continues to move across the surface of the sensor 210, additional readouts are then performed at consecutive readout time instances t=2, 3, 4 and 5 as further illustrated in FIG. 3A, and additional sets of pixel frame data 350-2, 350-3, 350-4 and 350-5 are obtained accordingly. At time instance t=5, the object 120 and the object points 122, 124 and 126 are no longer visible by the sensor 210, and the output set of pixel frame data 350-5 is thus once again “empty” (within the meaning as defined above). It should be noted in all of the non-empty sets of pixel frame data 350-1, 350-2, 350-3 and 350-4, there is motion blur of the object 120 caused by the object point projections ending up being registered as photons by multiple lines of sensor pixels 220 within each readout interval, as indicated by the symbols (circle, star and square, respectively) representative of the object points 122, 124 and 126 appearing on multiple pixel lines in each of the sets of pixel frame data 350-1, 350-2, 350-3 and 350-4.

It should be noted that in other envisaged examples, the object 120 may not necessarily move with constant speed. In such a case, it is envisaged that the readout frequency may be adjusted accordingly such that it e.g. changes between different consecutive pairs of readout time instances. For example, if the object 120 is accelerating, the distance (in time) between readout time instances t=0 and t=1 may be longer than the distance (in time) between readout time instances t=1 and t=2, etc. Likewise, if the object 120 is instead slowing down, the distance (in time) between readout time instances t=0 and t=1 may be shorter than the distance (in time) between readout time instances t=1 and t=2, etc. As long as information about the speed of the object 120 relative to the sensor 210, or at least information about the speed of the projection of an object point on the sensor 210, is available, it is envisaged that the readout frequency can be made time-varying and adapted such that between each pair of consecutive readout time instances t=i and t=i+1, the projection of the object point on the sensor 210 has time to move (i.e. moves) a distance corresponding to K lines of sensor pixels 220 (or K sensor pixels in the scanning direction, if e.g. there are no well-defined lines, if the sensor 210 is one-dimensional, etc.). In yet other examples, also K may change between consecutive pairs of readout time instances, based on e.g. how fast the object point projection is moving over the sensor 210.

In a similar fashion, FIG. 3B schematically illustrates another example (also without binning, B=1) but wherein the readout frequency is adjusted such that between two consecutive readout time instances, the projection of the points 122, 124 and 126 has time to move a distance across the sensor 210 corresponding to/spanned by three lines of sensor pixels 220, i.e. K=3. As can be seen in the various sets of pixel frame data 350-1, 350-2 and 350-3, there is additional motion blur caused by the projections of the object points 122, 124 and 126 being detected in not two but three lines of sensor pixels 220 during each readout interval.

FIG. 3C schematically illustrates yet another example wherein K=2, but wherein there is now binning of the lines of sensor pixels 220 before generating the sets of pixel frame data 350-0 to 350-5. In this particular example, the binning B corresponds to the number of lines of sensor pixels 220 visited by each object point 122, 124 and 126 projection during each readout interval, in that every two lines of sensor pixels 220 are combined to form a single pixel line in the corresponding set of pixel frame data. Phrased differently, B=K=2. As mentioned earlier herein, there may not necessarily be multiple sensor pixels 220 in each line, and/or one sensor pixel 220 may be considered in each line. Binning is however still possible, as it may e.g. be possible to combine every two sensor pixels 220 (in a one-dimensional sensor 210) to form an equivalent single-pixel output (B=2), every three sensor pixels 220 to form an equivalent single-pixel output (B=3), etc. In general, to illustrate the concept underlying the present disclosure, considering a plurality of sensor pixels 220 arranged in the scanning direction y is sufficient, independent of whether there are more sensor pixels 220 in the whole sensor 210, such as multiple sensor pixels 220 in each of a plurality of lines of sensor pixels 220 and similar.

In particular, in this example, binning is achieved by, at each readout time instance t, combining the data in the lines S1 and S2 to form the pixel line L1t; the data in the lines S3 and S4 to form the pixel line L2t; and the data in the lines S5 and S6 to form the pixel line L3t. The number of pixel lines in each set of pixel frame data is thus lower than the total amount of lines of sensor pixels 220. Such binning may e.g. be performed by adding the values of both lines in the sensor readout data 240 on a per-pixel basis, such that e.g. the first element of the pixel line L1t is obtained by adding together the first elements (pixels) of the lines S1 and S2; the second element of the pixel line L1t is obtained by adding together the second elements (pixels) of the lines S1 and S2, and so on for all elements (pixels) of the first pixel line L1t, and similar for the other pixel lines L2t and L3t. Generally speaking, with a binning defined by B, the sets of pixel frame data may be formed such that each pixel line Lit is found from a combination of the lines S[(i−1)×B]t, S[(i−1)×B+1]t, S[(i−1)×B+2]t, . . . , S[(i−1)×B+B]t=S(i×B)t, where with “combination” is meant that elements in different lines corresponding to a same pixel in each line are e.g. added together or somehow combined using any mathematical operation. For example, in addition to (or in combination with) using plain addition, an element of a particular pixel line Lit can be formed by weighting the corresponding sensor pixel values in the two or more lines of sensor pixels 220 (either uniformly or by applying different weighting indices to different pixel values), by taking an average, a mean, a min/max, some logarithmic or exponential combination, or in accordance with any other suitable linear or non-linear mathematical function or strategy (such as based on the use of machine learning/artificial intelligence) for how to combine values from multiple lines of sensor pixels 220 (or just from multiple sensor pixels 220) as part of the binning operation.

FIG. 3D schematically illustrates yet another example, but wherein K=3 and B=3, i.e. each projected object point has time to move a distance spanned by three lines of sensor pixels 220 during each readout interval, and the lines of the sensor readout data 240 are binned such that every three lines are added together as described above.

In all examples including binning, there may of course also be binning performed also in the x-direction (if there are more than a single sensor pixel in each line), i.e. such that the number of data elements in each pixel line is reduced to fewer data elements than there are sensor pixels 220 in each line of sensor pixels 220. For example, every pair of neighboring pixels may be combined (e.g. added, weighted, min'd/max'd, or combined using any other suitable mathematical function or strategy, using also e.g. machine learning/artificial intelligence) together to create a single data element, every three neighboring pixels may be combined together to create a single data element, etc. In other examples, such as those shown so far, binning is performed in the y-direction.

FIGS. 3E schematically illustrates yet another example, and serves to illustrate a situation in which the “binning-index” B does not match the “sensor line skipping-index” K. Here, the sensor 210 is assumed to have two additional lines SL7 and SL8 of sensor pixels 220, and the corresponding sensor readout data 240 thus defines two additional lines S7 and S8. Here, the movement of the object 120 is quicker, such that the projection of the points 122, 124 and 126 on the surface of the sensor 210 is such that a distance spanned by four lines of sensor pixels 220 are covered during each readout interval. Binning is performed with B=2, meaning that four pixel lines are created for each set of pixel frame data 350-0 to 350-3 (where sets of pixel frame data possibly generated after the projection of the points 122, 124 and 126 has left the sensor 210 are not shown).

FIGS. 3F and 3G each schematically illustrates various examples wherein the movement of the object 120 relative the sensor 210 is in the opposite direction, e.g. along a “reverse scanning direction” of the sensor 210. Here, the object 120 and the projections of the points 122, 124 and 126 first arrives at the first line SL1 and exits the sensor at the last line SL6. FIG. 3F illustrates an example with K=2 and no binning (B=1), while FIG. 3G illustrates an example with K=2 and binning of every two lines in the scanning direction of the sensor (B=2).

In the Figures related to binning, the relative sizes of the various symbols used in the illustrated sets of pixel frame data indicates how many photons from the object point that is included in each resulting “bin”. For example, in FIG. 3C, the square in line L32 is larger as the corresponding point 126 has been detected both on lines S5 and S6 of the sensor since the last readout, while the star symbol in line L32 is smaller due to the corresponding point 124 having been detected in line S5 since the last readout.

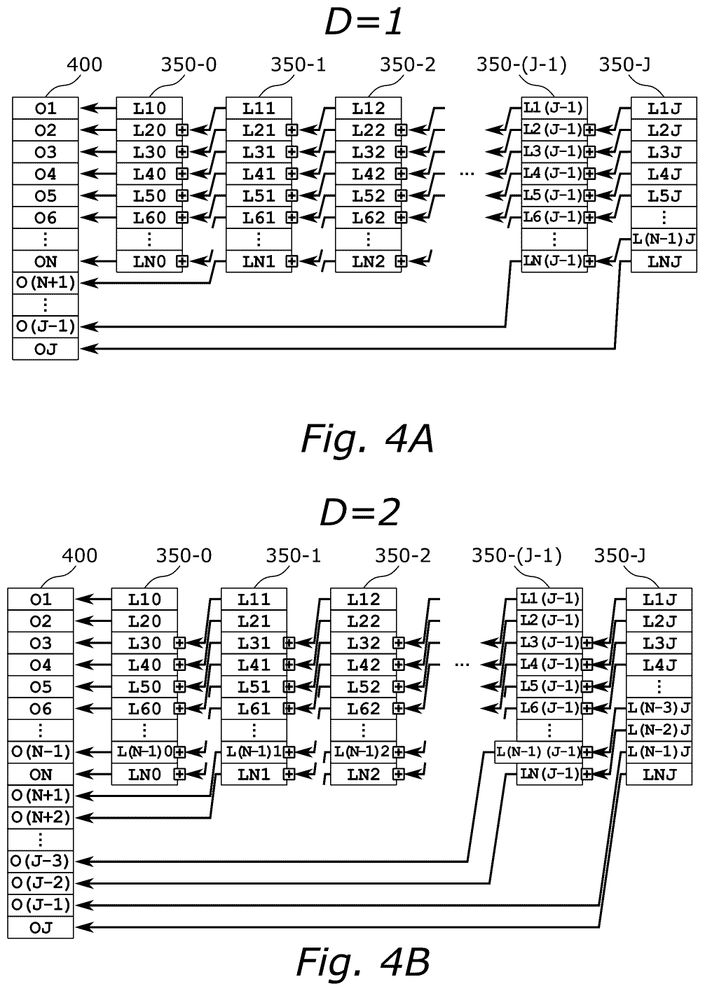

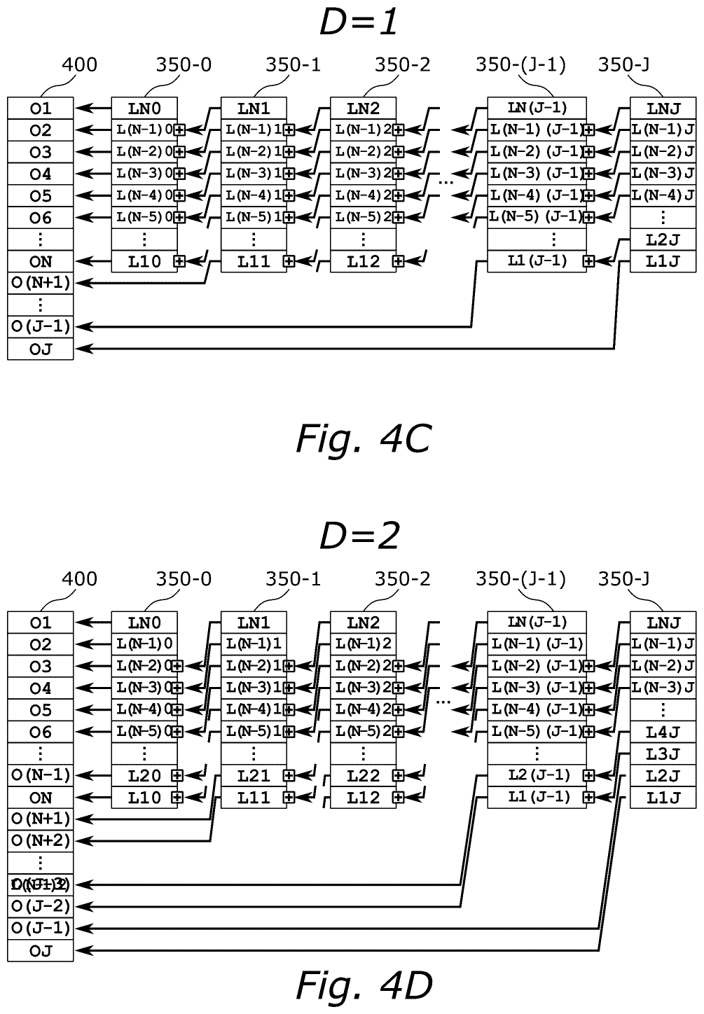

A second main idea underlying the present disclosure is that after having reduced the readout frequency such that the object point-projection has time to move (i.e. moves) a distance larger than a spacing/pitch of the sensor pixels in the scanning direction (i.e. K>1, such that K≥2), the resulting sets of pixel frame data obtained at different readout time instances are to be combined such that repeated readouts are accumulated over time. This includes to combine (e.g. add, weigh, etc.) data elements from different sets of pixel frame data such that the one or more adjacent sensor pixels associated with one data element are not the same one or more adjacent sensor pixels associated with the other data element. If using lines of sensor pixels, this means that data for one or more sensor pixel lines provided in one set of pixel frame data are to be combined with data for one or more other/different sensor pixel lines of the other set of pixel frame data. Phrased differently, data obtained from one or more first (lines of) sensor pixels capturing a particular point of the object at the first time instance is combined with data from one or more second (lines of) sensor pixels capturing the particular point of the object at the second time instance, wherein the first and second (lines of) sensor pixels are spatially offset in the scanning direction by a distance corresponding to the distance travelled by the object point-projection on the sensor 210, e.g. in accordance with K. For example, when pixel lines of two sets of pixel frame data corresponding to consecutive sensor readout time instances are added, the one or more consecutive lines of sensor pixels 220 from readouts of which a pixel line of one of the two sets of pixel frame data is formed are each spatially offset (in the scanning direction) more than one line of sensor pixels 220 from the one or more consecutive lines of sensor pixels from readouts of which a pixel line of the other one of the two sets of pixel frame data is formed. This concept will now be exemplified in more detail with reference also to FIGS. 4A, 4B, 4C and 4D.

FIG. 4A schematically illustrates how repeated readouts are accumulated over time where pixel lines that are to be added are such that the corresponding one or more lines of sensor pixels associated with one pixel line are offset an integer number D=1 from the one or more lines of sensor pixels associated with the other pixel line. Herein, that each pixel line (in a set of pixel frame data) may be associated with, or obtained based on readouts from, more than one line of sensor pixels 220 is a consequence of that readouts from multiple lines of sensor pixels 220 may be combined to form a single pixel line in a set of pixel frame data, e.g. as a result of binning as discussed earlier herein. If no binning is performed before accumulating the rows, each pixel line will be associated with, or originate from readouts of, a single line of sensor pixels 220.

In the example of FIG. 4A, it may be assumed that the projection of the object points of the object 120 moves in the forward scanning direction, e.g. as illustrated and exemplified in any of FIGS. 3A to 3E. The arrows indicate how data is transferred from one pixel line to another, and the squares with a plus-symbol indicates that the data incoming from another pixel line to the pixel line having the square with the plus-symbol is added to (or otherwise somehow combined with) the data of the latter. In this way, data from pixel lines associated with different readout time instances are accumulated, and the result is forwarded to form an accumulated image 400. The image 400 may e.g. have a number of lines O1, O2, . . . , OJ, where J depends on the total number of different readout time instances used to scan the object 120. For example, if using the notation previously introduced herein, where the first readout time instance is t=0 and the last readout time instance is t=T, the integer number/may equal J=T+1. As may be seen in FIG. 4A, one row of the image 400 will be output for each readout time instance. At the end of the first readout time instance t=0, the data of the first pixel line L10 in the first set of pixel frame data (such as 350-0) is not combined with any other pixel line data and instead outputted as-is to form the first line O1 of the image 400. The second line O2 of the image 400 will be formed by accumulating data from both the second pixel line L20 of the first set of pixel frame data (e.g. 350-0) and the first pixel line L11 of the second set of pixel frame data (e.g. 350-1). Continuing in this way, it can be seen that for D=1, the third line O3 of the image 400 is found by accumulating data from the third pixel line L30 of the first set of pixel frame data 350-0, the second pixel line L21 of the second set of pixel frame data 350-1, and the first pixel line L13 of the third set of pixel frame data (e.g. 350-2), and so on. After having performed a number of readouts equal to the number N of lines of sensor pixels 220 in the sensor 210, the number of pixel lines from different sets from which data is accumulated to form the remaining new lines of the image 400 will settle at a number equal to N. For example, to generate line ON of the image 400 will use accumulation of data from pixel lines LN0, L(N−1)1, L(N−2)2, . . . , L1(N−1); and generating line O(N+1) of the image 400 will use accumulation of data from pixel lines LN1, L(N−1)2, . . . , L1N. In this example, the last line OJ added to the image 400 will include data obtained from pixel line LNJ, i.e. from the last set of pixel frame data (e.g. 350-J) captured/obtained during the last readout of the sensor 210.

FIG. 4B is an example using the same premises as the example shown in FIG. 4A, but where the offset is instead equal to D=2. Phrased differently, in this example, two pixel lines associated with adjacent readout time instances are to be selected such that the offset is D=2. Thus, for each time instance, two lines will be added to the image 400. After the first readout time instance t=0, data from pixel lines L10 and L20 are not combined with any other pixel lines, but added directly as lines O1 and O2. At the next readout time instance, t=1, line O3 of the image 400 is found by accumulation of data from pixel lines L30 and L11, while line O4 of the image is found by accumulation of data from pixel lines L40 and L21. Likewise, line O5 of the image 400 is found by accumulation of data from pixel lines L50, L31 and L12, and line O6 of the image 400 is found by accumulation of data from pixel lines L60, L41 and L21. After having performed N readouts, the number of rows from which data is accumulated to create each new row of the image 400 settles at a number equal to N/2. Line O(N−1) of image 400 will use accumulation of data from pixel lines L(N−1)0, L(N−3)1, . . . , L1(N/2); and line ON of image 400 will use accumulation of data from pixel lines LN0, L(N−2)1, . . . , L2(N/2). The last two lines O(J−1) and OJ added to the image will include data from pixel lines L(N−1)) and LNJ, respectively, i.e. from the last set of pixel frame data obtained at the last readout of the sensor 210.

Generally speaking, for a particular D, each readout of the sensor may thus add D new lines to the image 400.

More generally, as envisaged herein, the lines (or data elements) of the various sets of pixel frame data 350-t are to be combined such that e.g. a first data element of a first set of pixel frame data and associated with one or more adjacent sensor pixels 220 is combined with a second data element of a second set of pixel frame data and associated with other/different one or more adjacent sensor pixels 220 than those of the first data element. If there is no binning (B=1), the data element (or line of data elements) of the first set of pixel frame data is associated with a sensor pixel (or line of sensor pixel) that is spatially offset a distance in the scanning direction y that equals the distance moved over the sensor 210 by the object point-projection, e.g. K times a distance between adjacent sensor pixels in the scanning direction. Likewise, if there is binning (B≥2), this still applies. In this case, the two or more adjacent (lines of) sensor pixels associated with the first data element will be combined with two or more adjacent (lines of) sensor pixels associated with the second data element, and on an individual-basis, the associated (lines of) sensor pixels are still spatially offset in the scanning direction by a distance equal to the distance moved over the sensor 210 by the object point projection. Phrased differently, the first (line of) sensor pixel of the bin associated with the first data element (or pixel line data) is offset such a distance from the first (line of) sensor pixel of the bin associated with the second data element, etc.

FIGS. 4C and 4D schematically illustrates similar examples to those of FIGS. 4A and 4B, respectively, but for the case when the projection of the object point(s) move in the reverse scanning direction of the sensor (as illustrated and exemplified in FIGS. 3F and 3G). As can be seen, the logic may be obtained by providing the pixel lines in each set of pixel frame data in reverse order, such that e.g. the first pixel line L1t in each set is interchanged with the last pixel line LNt of the same set, the second pixel line L2t is interchanged with the next-to-last pixel line L(N−1)t, etc. Other possible solutions are of course also available, as long as the end result is the same. In particular, here and in all other envisaged examples herein, care should preferably be taken such that pixel lines formed by data obtained by readouts of photons originating from a same point of the object 120 are accumulated together.

Generally herein, a general principle is that in case no binning is performed, combination/accumulation of data from various pixel lines/data elements of different sets of pixel frame data should follow the principle that B=K. Using FIG. 3A as an example of such a situation, it can be seen that by selecting D=2, data will be accumulated from e.g. pixel lines L51, L32 and L13 which all correspond to detection of photons originating from a same feature/point 122 of the object 120 (as illustrated by the circle symbol being present in all of these pixel lines). Likewise, data will be accumulated from pixel lines L61, L42 and L23, all including data from the detection of photons originating from the point 124 of the object 120, as seen by the star symbol appearing in all of these pixel lines, etc. There will of course be some motion blur remaining, but it is here considered a reasonable tradeoff in exchange for a reduced ratio of sensor readout time to time spent on detecting/counting photons due to the reduced readout frequency.

Also herein, if binning is used, the principle is that D may not necessarily match K. For example, D=1 may be used at least as long B=K>1, e.g. when K=2 and B=2such as illustrated and exemplified in e.g. FIG. 3C. For example, it may be seen that using D=1 will as desired accumulate data from pixel lines L31, L22 and L13 all including the point 122, etc. Also here, some motion blur will still remain. However, using binning before accumulating the pixel row data may have, as will be explained in more detail later herein, the added benefit that as D is not required to be equal to K, and may instead be reduced to e.g. D=1, a reduction (or more efficient use) of hardware resources can be achieved. Generally herein, it is however considered that when binning is used, the binning is such that B=K.

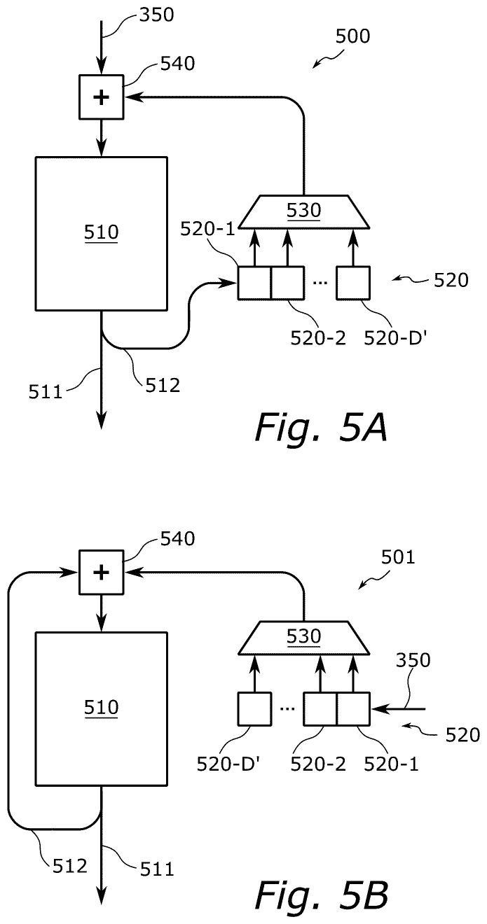

Herein, it is envisaged that the various accumulations of the pixel lines (or data elements) in the various sets of pixel frame data may be performed by the device 300, e.g. as part of a data post-processing unit/part of the device 300. Examples of such post-processing units will now be described in more detail with reference also to FIGS. 5A-5D.

FIG. 5A schematically illustrates various functional blocks of an example post- processing module 500 of the device 300. The module 500 includes a memory 510 in which data for a plurality of pixel lines can be stored and accessed. The memory 510 may also be referred to as a “data structure” or similar, and may be e.g. a general memory, a queue, a stack, list, an array, or other data structure suitable for storing and accessing data for pixel lines (or sums of data from multiple pixel lines). The module 500 further includes one or more line delay elements 520-1 to 520-D′ (also referred to using just the numeral “520”), where e.g. a total number of such elements 520 matches a desired D, i.e. such that D′=D. The line delay elements 520 are each configured to store data corresponding to one pixel line, and may e.g. be implemented as a RAM buffer, one or more logical elements or by other suitable components. When a line delay element 520 receives new pixel row data to store, the previously stored pixel row data is provided as output from the line delay element 520. If more than one line delay element 520 is present/actives, the plurality of line delay elements 520 are connected together in a daisy-chain fashion such that an output of the first line delay element 520-1 is provided as input to the second line delay element 520-2, etc. Thus, as the first line delay element 520-1 receives new pixel line data to store, it outputs its previously stored pixel line data (if any) as input to the next line delay element 520-2, and so on. The output of each line delay element 520 is also provided to a multiplexer 530. The multiplexer 530 selects an output from a particular one of the one or more line delay elements 520, and provides this selected output to an adder 540, which adds (on a per-pixel element basis) the pixel line data (if any) obtained from the multiplexer 530 to pixel line data 350 obtained from a set of pixel frame data. Part or the whole of the daisy-chain structure may be implemented as a data structure, such as e.g. a queue, ring buffer, or similar, in computer (e.g. RAM) memory.

Here, it is assumed that the post-processing part receives new sets of pixel frame data as they are readout from the sensor 210, either directly or after having performed (pre-) binning as discussed herein, and processes each set of pixel frame data on a line-by-line basis (or on an element-by-element, in case no lines are defined/used). By suitable configuration of the memory 510 and number of line delay elements 520 that are active, pixel line data/data elements from earlier sets of pixel frame data may iteratively be combined with pixel line data/data elements from more recent sets of pixel frame data, in order to generate the desired accumulation of pixel line data/data elements as exemplified in FIGS. 4A to 4D. Depending on whether the object 120 moves in the forward or reverse scanning direction of the sensor 210, the pixel lines/data elements of the sets of pixel frame data 350 can be inserted starting from the top of the sensor 210 (i.e. such that line L1t is processed before line L2t, and so on), or inserted starting from the bottom of the sensor 210 (i.e. such that line L6t is inserted before L5t, and so on).

Generally, the module 500 may be operated on a step-by-step basis, using e.g. a clock signal (such as a pulse train or similar). For each “tick”, data (i.e., pixel line data/data elements) flows one step forward in accordance with the flowchart indicated in FIG. 5A. For example, each tick, a new pixel line/data element is read from a current set of pixel frame data, and provided to the adder. The adder also receives pixel line data/data elements from the last active/selected line delay element 520, and adds this to the new pixel line/data element. The result (i.e. the sum) is the inserted as a new line/element into memory 510. For each new line/element added to the memory 510, it is checked whether a previously added line/element is now ready to be extracted from the memory (path 511) and provided to e.g. form a new line/element of the image 400, or if the previously added line/element will form part of a further accumulation of pixel line data/data elements, in case the line/element is instead provided (path 512) as input to the first line delay element 520-1. If there are multiple line delay elements 520, whatever is previously stored in each of the line delay elements 520 is sent to a next line delay element 520 in the daisy-chain, and the output of the last line delay element will thus be used as input to the adder 540 and added to the following pixel line. As the last pixel line of a particular set of pixel frame data is processed (i.e. sent to memory 510), a next set of pixel frame data is made ready for being processed. As used herein, the term “line delay element” may of course also be referred to as a “data element delay element”, or just “data delay element”, etc., if no lines are defined.

FIG. 5B schematically illustrates another envisaged example post-processing module 501 that may be configured to perform a same task as the post-processing module 500 just described with reference to FIG. 5A. Here, the new pixel lines/data elements from the sets of pixel frame data 350 are instead fed into the daisy-chain (or the at least one line delay element 520-1), and the output of the last line delay element 520 is provided as input to the adder 540. In case there is a previously stored line/element in the memory 510 that is now ready for output (that is, this particular line/element will not take part in any further accumulation of pixel line data/data elements from different sets of pixel frame data), this line/element is output (path 511) to e.g. form a new line/element of the image 400. If it is decided that the line/element will instead be part of further accumulation, the line/element is output from the memory and sent (path 512) as input to the adder 540, such that it is added to whatever line/element received from the multiplexer 530.

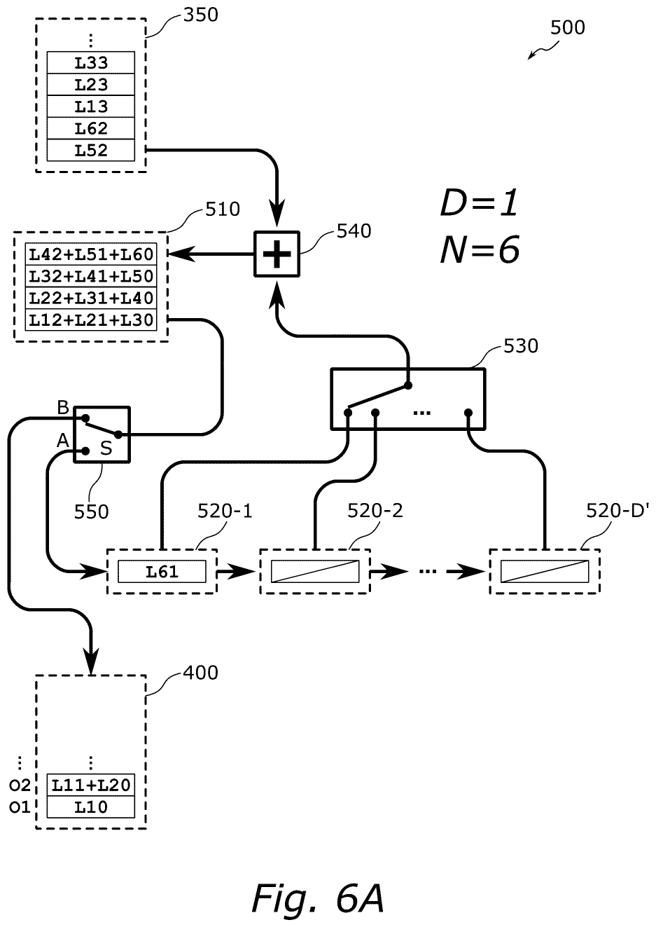

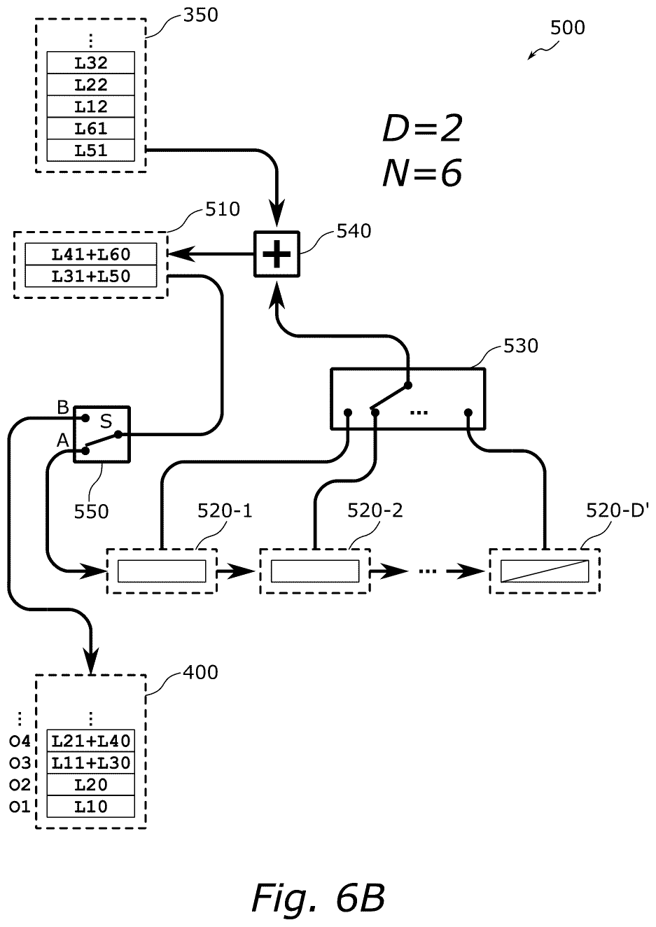

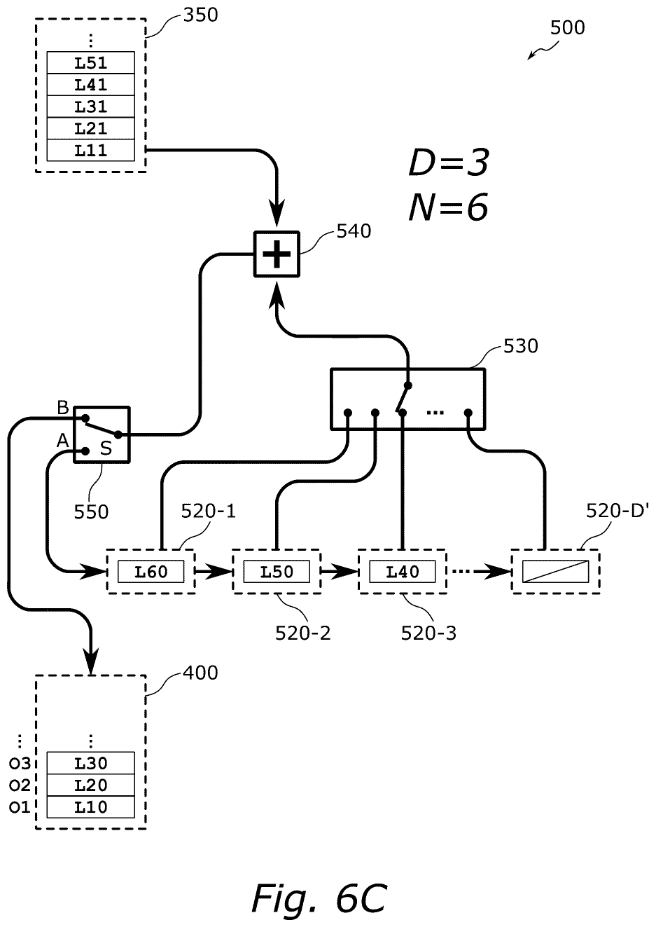

A more detailed example of the inner workings of the post-processing module 500 will now be described with reference also to FIGS. 6A, 6B and 6C.

FIG. 6A schematically illustrates one example of how the module 500 can be implemented/configured. Here, the memory 510 is provided as a first-in, first-out (FIFO) data structure, with a number of total memory lines/elements equal to N−D×2. The example of FIG. 6A is provided assuming that the sensor 210 has six lines of sensor pixels 220 (as used in the examples of e.g. FIGS. 3A-3D, 3F and 3G). The module 500 is configured for the case D=1, and the number of memory lines in the memory 510 thus equals four. As D=1, the first line delay element 520-1 is active, e.g. the multiplexer 530 is operated such that it picks the output from the first line delay element 520-1 as its output, while any content of the other line delay elements 520-2 to 520-D′ is ignored. FIG. 6A illustrates a particular snapshot of time, wherein processing of a set of pixel frame data associated with readout time instance t=2 is almost completed. So far, four of the six pixel lines of this set of pixel frame data has already been proceed, and the remaining two pixel lines are waiting to be inserted into memory 510. It is noted that the bottom memory line of the memory 510 is ready to be output to form a new line of the image 400, which is used to trigger a (logical) switch/selector 550 such that once the next pixel line to be processed (here L52) is added to the memory, the line corresponding to an accumulation of data from pixel lines L12+L21+L30 will be pushed to the image 400 instead of to the first line delay element 520-1. The first line-delay element 520-1 currently holds data for the pixel line L61, and the data for this pixel line will thus be added to the pixel line L52 before the sum thereof is added to the memory 510 during a next “tick” of the module 500. By comparing the expected output from the module 500 as more ticks are generated with e.g. FIG. 4A, it can be seen that the desired accumulation of data from pixel lines from different sets of pixel frame data will follow as a result.

FIG. 6B schematically illustrates another example of how the module 500 can be implemented/configured, where D=2 and N=6. As a consequence, the multiplexer 530 is now configured to select as its input the output from the second line delay element 520-2 instead of the first line delay element 520-1. The number of memory lines/elements in the memory 510 is reduced to two, according to the formula N−2×D as described with reference to FIG. 6A. At the particular snapshot illustrated in FIG. 6A, it is seen that the line of the memory 510 corresponding to L31+L50 is not yet ready to be output to the image 400, and the switch 550 is therefore configured (in position “B”) such that the line corresponding to L31+L50 will instead, once the next new pixel line L51 is processed by sent as input to the first line delay element 520-1 (which is currently empty, as the switch 550 was previously configured in position “A” as part of outputting what is now line O4 of the image 400 (i.e. the line L21+L40). As the second line delay element 520-2 is also empty (due to the switch being in position “A” also when outputting what is now row O3 of image 400), no data will be added to the pixel line L51 at this step. By comparing the current state of the module 500 and the expected future flow of data as more ticks are generated with FIG. 4B, it can be seen that the performance of the module 500 in the example of FIG. 6B also is as expected.

FIG. 6C schematically illustrates yet another example of how the module 500 can be implemented/configured, when D=3 and N=6. In this case, there is no need for the memory 510, as there is, in this particular example, one line output to the image 400 for each new pixel line. For example, the new line L11 will be added to L40 (currently stored in a third line delay element 520-3) and directly output as a new line O4 of the image 400. The next new line L21 will be added to L50 (which is currently stored in the second line delay element 520-2, but will be shifted to the third line delay element 520-3 when the pixel line L11 is processed) and directly output as a new line O5 of the image 400, and so on. Generally, with given values for N and D, the number of memory lines of the memory 510 is, as explained earlier herein, given e.g. by the expression N−2×D. It is noted that the memory 510 may of course still be configured/able to store more lines, but that the readout mechanism may then be adjusted such that lines that are currently not utilized are ignored, or similar. This allows the flexibility of using a same memory for multiple situations, with various values of N and D.

How to configure the switch 550 may for example be determined by checking whether the current bottom memory line/element of the memory 510 includes data associated with one of the D first lines/elements of a set of pixel frame data that is currently being processed. For example, in the example of FIG. 6A, the switch was changed to position “B” once it was detected that the bottom memory line of the memory 510 was the first (D=1) pixel line of the currently processed set of pixel frame data, and otherwise to position “A”. In the example of FIG. 6B, the switch was changed to position “B” once it was detected that the bottom memory line of the memory 510 was either the first or second (D=2) pixel line of the currently processed set of pixel frame data, and otherwise to position “B”, etc.

The example post-processing module 501 may of course be implemented similarly to what is shown in FIGS. 6A-6C, by reconfiguring the various functional entities (such as the memory 510, one or more line delay elements 520, the multiplexer 530 and adder 540) as indicated by the flowchart of FIG. 5B.

It is envisaged that both of the post-processing modules 500 and 501 can be implemented using e.g. the processing circuitry 310 of the device 300 as envisaged herein. Here, the term “processing circuitry 310” is assumed to include software, hardware, or a combination of both to perform the various operations of the post-processing modules 500 and 501. It should also be noted that due to symmetry considerations, the two modules 500 and 501 may produce a same output depending on whether the pixel lines of the sets of pixel frame data are provided starting from the top or bottom of the sensor 210. In principle, one of the modules 500 and 501 provided with pixel lines in one order should generate a same output in the end as the other one of the module 500 and 501 provided with pixel lines in the opposite order.

For example, the situation of FIGS. 4C and 4D, corresponding to when the object 120 moves in the reverse scanning direction of the sensor 210, may be handled by e.g. the post-processing module 500 by instead feeding the pixel lines in reverse order, such that (for a set of pixel frame data associated with readout time instance t) pixel line L6t is processed before L5t, and so on.

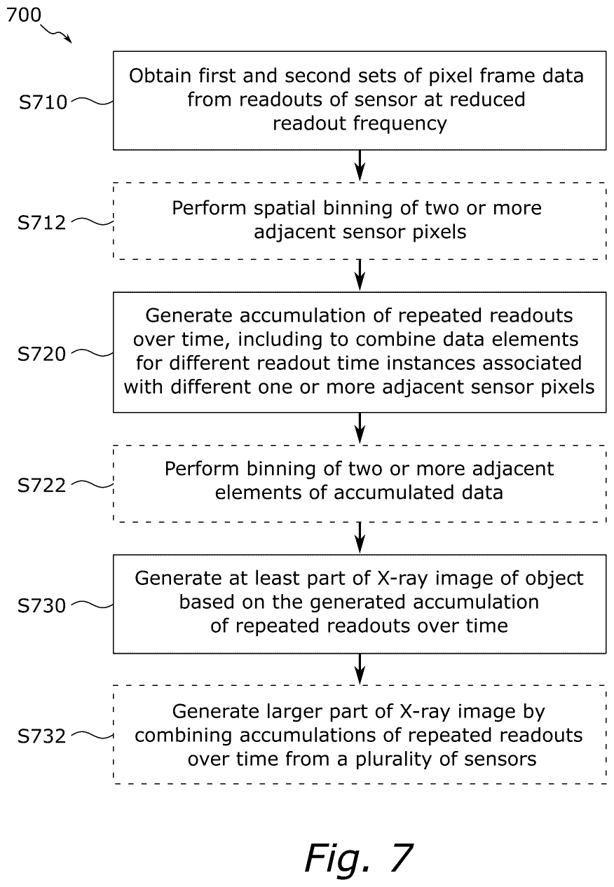

A method for generating an X-ray image using one or more multiline X-ray sensors (such as any sensor 210) will now be described in more detail with reference also to FIG. 7.

FIG. 7 schematically illustrates a flowchart of an exemplary method 700 according to the present disclosure. As part of a first operation S710, the method 700 includes obtaining at least first and second sets of pixel frame data from a sensor having a plurality of sensor pixels spatially offset by the first distance in the scanning direction. Each set of pixel frame data includes data representative of a plurality of data elements each associated with one or more (such as B) adjacent sensor pixels (of the plurality of sensor pixels). Phrased differently, each set of pixel frame includes data for a plurality of data elements (such as e.g. those of pixel lines, e.g. L1t, L2t, . . . , LNt) formed from readouts of a first number (e.g. B) of (lines of) sensor pixels 220 (such as SLi, SL(i+1), . . . , SL(i+B−1)) of the sensor 210. The readout frequency is such that between the first and second readout time instances, e.g. between t=t′ and t=t′+1, a projection on the sensor 210 of a point (such as e.g. 122, 124 and/or 126) of the imaged object 120 has had time to move a second distance larger than the first distance between the sensor pixels in the scanning direction, e.g. a distance spanned by K consecutive (lines of) sensor pixels (e.g. from line SLj to SL(j+K)) in the scanning direction of the sensor 210, where i.e. K>1 (such that K≥2).

The second distance may be smaller than a distance spanned by all of the plurality of sensor pixels (e.g. smaller than N times the first distance, if there are N sensor pixels in total in the plurality of sensor pixels, such that K<N).

As part of a second operation S720, the method 700 includes combining a first data element of the first set of pixel frame data with a second data element of the second set of pixel frame data, wherein the first and second data elements are associated with different sensor pixels. This as part of generating an accumulation of the repeated readouts over time. For example, if assuming that the first and second data elements are associated with sensor pixels in two pixel lines Lit and Li′t′, respectively, that are to be combined (e.g. added, as part of the accumulation process), it is not the case that t=t′ or that i=i′. Instead, if e.g. t′=t+1, the “pixel line indices” i and j are selected such that j=i±D. If no binning is used (B=1), D is equal to or greater than two. If binning is used (B>1), D may be equal to or greater than one.

As part of a third operation S730, the method 700 includes generating at least part of an X-ray image (such as image 400) of the object 120 based on the combination of the first data element and the second data element (e.g. on the generated accumulation of the repeated readouts over time), as has been illustrated in e.g. FIGS. 4A-4D and 5A-5C.

In some examples, the method 700 may include an optional operation S712 that includes performing spatial binning of the (lines of) sensor pixels 220 before performing the accumulation of data elements/pixel line data (i.e. the combining of the first and second data elements). The spatial binning is then at least in the scanning direction of the sensor 210, but may optionally include spatial binning also in the direction transverse to the scanning direction of the sensor 210, i.e. binning of one or more sensor pixels in a same line of sensor pixels 220. Here, “binning of sensor pixels” means that their outputs are combined to form an equivalent larger pixel transverse the scanning direction, such as described earlier herein (by e.g. addition, weighted addition, median value or any other mathematical function or algorithm for pixel binning). Likewise, “binning of lines of sensor pixels” means that the output of corresponding sensor pixels in all binned lines are combined to form an equivalent of a line of sensor pixels that are larger in the scanning direction. Binning is performed pair- or n-tuple-wise, e.g. such that B (lines of) sensor pixels are binned together, such that e.g. lines SL1 to SL(1+B−1) are binned to form a first pixel line L1; lines SL(1+B) to SL(1+2B−1) are binned to form a second pixel line L2; and similar, such that lines SL(i) to SL(i+B−1) are binned to form a pixel line Li, where i=1,2, . . . , N/B. Within the realm of the present disclosure, such binning may be referred to as “pre-binning” (as performed before e.g. the post-processing modules 500 and 501).

In some examples, the method 700 may include an optional operation S722 that includes performing binning of two or more elements/lines of the accumulated data, such as e.g. two or more elements/lines Oj of the image 400. As before, such binning includes combining data from corresponding elements of the image 400 to form a new element (or e.g. combining elements of a line of the image 400 to form a new line of the image 400), wherein such binning may be performed in the scanning direction (i.e. data elements from different lines are added if lines are defined), transverse the scanning direction (i.e. data elements of a same line are added if lines are defined), or both. This binning may be referred to as “post-binning” (as performed after e.g. the post-processing modules 500 and 501).

In some examples, the method 700 may include an optional operation S732 that includes generating a larger part of the X-ray image by combining accumulations of repeated readouts over time for many sensors 210. Phrased differently, operations S710, S720 and S730 may be performed in parallel for sensor pixel data read out from each sensor). For example, referring back to FIG. 2, accumulations as described herein may be performed for a plurality (such as all) of the sensors 210, and the results (e.g. image 400) may be combined to form a larger X-ray image where each part of the larger X-ray image corresponds to the readouts from one of the sensors 210.

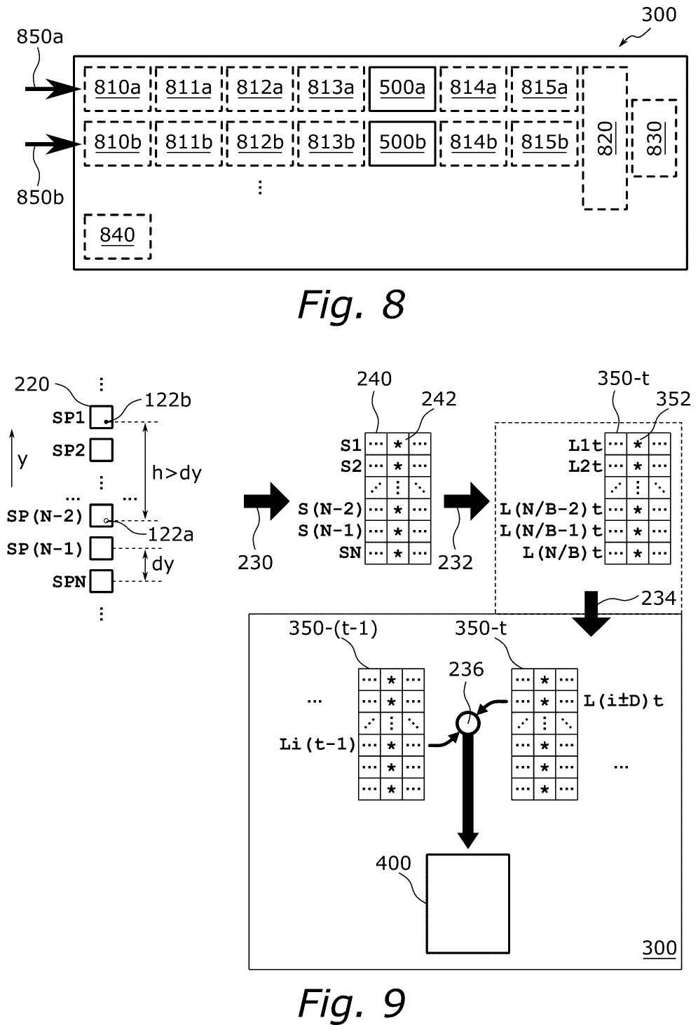

Various envisaged implementations of the device 300 will now be described in more detail with reference also to FIG. 8.

FIG. 8 schematically illustrates various examples of a device 300 in terms of a number of functional modules 810a-b, 811a-b, 812a-b, 813a-b, 814a-b, 815a-b, 820, 830, 840 and 500a-b. It is envisaged that the device 300 includes at least the functional module 500a, corresponding to the post-processing module 500 (or 501) described with reference to e.g. FIGS. 4A, 5B and 6A-6C, and that some or all of the other modules may be optional.

The module 500a, i.e. the post-processing module 500 (or 501) is configured to perform at least the operation S720 of the method 700 related to the generating of the accumulation of data elements/pixel line data from sets of pixel frame data associated with different readout time instances (i.e. at least the combination of the first and second data elements), following e.g. the flow of data and various functional entities shown in FIGS. 5A and 5B. The module 500 may also be referred to as e.g. a “post-processor”, “time delay summator”, “TDS module”, “TDS processor”, or similar.

The device 300 may in some examples optionally also include an IO module 810a responsible for obtaining the data readout from the sensor 210, and e.g. be configured to perform operation S610 of the method 600. The IO module 810a may also be responsible for e.g. receiving one or more configuration parameters pertinent to the readout and/or control of the sensor 210, as well as to other user-configurable parameters of the device (such as the readout mode, whether scanning is performed in the forward or reverse scanning direction, the values for D, K and/or B, etc. The module 810a may also be referred to as e.g. a “data input/output transceiver”, “IO transceiver”, or similar.

The device 300 may in some examples optionally also include a sorting module 811a responsible for e.g. sorting the received data on a form suitable for being processed by the post-processing module 500a. For example, if the readout data from a sensor 210 having a plurality of lines of sensor pixels is not obtained on a line-by-line basis, the sorting module 811a may be configured to convert the readout data to such a line-by-line format, and e.g. sort the data such that for example the lines are presented to the post-processing module 500a on a “from top-to-bottom of the sensor” format, or e.g. on a “from a bottom-to-top of the sensor” format, as described earlier herein. The sorting module 811a may also be referred to as e.g. a “sorter”, “data transformer”, or similar.

Generally herein, the order in which the data from the readout of a sensor arrives at the device may be arbitrary. For example, all data elements may be processed in any temporal order or may e.g. be divided between multiple processing units/circuitry. Any sorting transformation that alters the order of data, but not the data itself, may be used if a corresponding transformation is applied also to the various operations disclosed herein. For example, there may be one or more technical reasons to use a non-temporally, non-spatially ordered data for more efficient use of e.g. FPGA-cells,-blocks or other components. For example, each data element may include a label indicative of to which particular sensor pixel (or to which particular sensor pixels) the data element is associated, and such labels may then be used to order the data elements in any desired state. The particular examples provided herein all, for clarity reasons, include data that appears to be sorted spatially, e.g. line-by-line or data element-by-element from for example top to bottom of a sensor, but it is envisaged that that may not necessarily be the case. As long as the end result is the same, namely that data elements from different sets of pixel frame data that has detected photons originating from a same object point are eventually at least partially combined, the present disclosure is relevant also for any particular ordering/sorting (or lack thereof) of the input data from the readout of the sensor(s).

The device 300 may in some examples optionally also include a blocking module 812a responsible for e.g. blocking one or more pixel lines from being further processed. For example, the blocking module 812a may be configured to remove e.g. one or more of the top pixel lines, or e.g. pixel lines associated with one or more of the top lines of sensor pixels 220 of the sensor 210, and/or similarly for one or more bottom pixel lines or lines of sensor pixels 220, and/or e.g. to remove (from each pixel line) elements associated with e.g. one or more sensor pixels on each side (e.g. left or right) of the sensor 210, and similar. This may be advantageous in that such lines or pixel elements may provide less useful data due to lying at a periphery of the sensor 210, and similar. The blocking module 812a may also be referred to as e.g. a “blocker”, or “masker”, or similar.

The device 300 may in some examples optionally also include a pre-binning module 813a responsible for e.g. performing the optional operation S712 of the method 700, as described earlier herein. The pre-binning module 813a may also be referred to as e.g. a “pre-binner” or similar.

The device 300 may in some examples optionally also include a first post-binning module 814a responsible for e.g. performing the optional operation S722 of the method 700, as described earlier herein. The first post-binning module 814a may also be referred to as e.g. a “first post-binner”, or similar.

The device 300 may in some examples optionally also include an image buffering module 815a, responsible for e.g. performing the operation S730 of the method 700, e.g. to collect the elements/lines output from the module 500a (and/or any subsequent modules) to build at least part of an X-ray image of the object 120, such as the image 400. The image buffering module 815a may also be referred to as e.g. an “image buffer”, or similar.

The device 300 may in some examples optionally also include a readout controlling module 840, responsible for e.g. controlling the readout frequency of the sensor 210, if not handled by any other module of the device 300. The readout controlling module 840 may also be referred to as e.g. a “readout controller” or similar.

As shown in FIG. 8, there may, in some examples of the device 300, also be one or more additional branches similar to the branch containing the modules 810a, 811a, 812a, 813a, 500a, 814a, and 815a. For example, if the detector 200 includes multiple sensors 210, there may be one such branch (such as a branch containing module 500b and optionally one or more of the modules 810n, 811b, 812b, 813b, 814b and 815b, each similar to their respective module indexed with the letter “a” instead of “b”, as described herein) for each sensor 210. In such a case, the device 300 may include a multiplexing module 820 responsible for either selecting accumulated data (such as image 400) from one of the modules as output from the device as the X-ray image of the object 120, or to e.g. combine such accumulated data (such as images 400) from a plurality (such as all) of the sensors 210 and branches of modules. The multiplexing module 820 may also be referred to as a “multiplexer”, “image combiner”, “accumulation data combiner”, or similar.

The device 300 may also, in some examples optionally also include one or more additional modules 830, including e.g. a second post-binning module/binner responsible for performing post-binning of the accumulated data if this is not already done by e.g. one or more first post-binning modules 814a,b, . . . , as described earlier herein.

In general terms, each functional module described above with reference to FIG. 8 may be implemented in hardware or in software. Preferably, one or more or all functional modules may be implemented by the processing circuitry 310, possibly in cooperation with the communications interface 330 and/or the storage medium/memory 320. The processing circuitry 310 may thus be arranged to from the memory 320 fetch instructions as provided by a functional module, and to execute these instructions and thereby perform any operations of the method 700 performed by/in the device 300 as disclosed herein. The device 300 may also be referred to as an “X-ray image generating entity”, “X-ray image generator”, “X-ray imaging control unit”, or similar.

Particularly, the processing circuitry 310 is configured to cause the device 300 to perform a set of operations, or steps, needed to perform all or part of the method 700 as described with reference to FIG. 7. For example, the memory 320 may store a set of operations, and the processing circuitry 310 may be configured to retrieve the set of operations from the memory 320 to cause the device 300 to perform the set of operations. The set of operations may be provided as a set of executable instructions. Thus, the processing circuitry 310 is thereby arranged to execute methods associated with generating one or more X-ray images of a (moving) object as disclosed herein, e.g. with reference to any one of the Figs. of the accompanying drawings.

It is to be noted that the device 300 envisaged herein is suitable for parallel processing of sensor data readout from multiple sensors 210 of a detector 200. This because as the initial spatial-blurring (caused by the reduction of the readout frequency) happens very early in the processing chain using low-complexity and less power-consuming operations and components closer to the temperature-sensitive sensor parts. For example, by reducing the readout frequency compared to the contemporary TDS solution, less data is read out from the sensor in total, at fewer readout intervals, which reduces the computational load and e.g. a heating of components closer to the sensor 210. In addition, the proposed architecture is also suitable for highly distributed processing of data, as the data readout from each sensor 210 can be processed individually for a major part of the overall processing chain. The reduced number of readout intervals reduces the total number of accumulation operations (by e.g. a factor K), as the number of input samples are e.g. halved if K=2. If there is a need for additional spatial binning, such as post-binning, this can be implemented later in the processing chain, where more complex processors or e.g. FPGAs can be used for more sophisticated image processing.

As has also been shown herein, the envisaged use of pre-binning can have the advantage that the number of line delay elements 520 can be reduced and still obtain multi-step TDS-like data. As implementing a line delay element in e.g. an FPGA consumes often valuable resources, the resources available for other tasks may thus be increased, or the overall requirements of the FPGA can be relaxed leading to e.g. a higher cost-efficiency. For example, if using a binning of B=2, it may be possible to use a single line delay element 520-1 instead of two, as B=2 allows to reduce D from two to one.

FIG. 9 finally schematically illustrates a more general working principle of the solutions envisaged herein, as well as an exemplary device 300. Here, it is not assumed that the sensor 210 necessarily includes lines of sensor pixels 220, but that the sensor has at least a plurality of sensor pixels 220 that are spatially offset from one another in the scanning direction y of the sensor. The offset is indicated by a first distance dy. It is further assumed that the offset may be different for one or more other sensor pixels 220 (if present) of the sensor than the sensor pixels 220 shown in FIG. 9. The empty circle 122a indicates a projection of the object point 122 on the sensor 210 at a first readout time instance, and the filled circle 122b indicates the projection of the object point 122 on the sensor at a second readout time instance (that may e.g. be consecutive to the first readout time instance). The second readout time instance may e.g. correspond to t, and the first readout time instance to e.g. t−1. Between the time instances t−1 and t, the projection of the object point 122 moves (or has at least had time to move) a second distance h that is larger than the first distance dy.

In FIG. 9, SP1, SP2, . . . , SPN denotes sensor pixels. During the readout operation 230, sensor readout data 240 is formed, including cells 242 for each sensor pixel 220. Each cell 242 may e.g. include a total photon count detected by the corresponding sensor pixel 220 during the last readout interval (i.e. between t−1 and t), and similar, in particular if the detector/sensor is photon counting detector/sensor. In a subsequent operation 232, the sensor readout data 240 is transformed into a set of pixel frame data 350-t, i.e. the set 350-tcorresponds to the readout of the sensor at time t. The set of pixel frame data 350-t includes data representative of a plurality of data elements 352 that are each associated with one or more adjacent sensor pixels of the plurality of sensor pixels 220. How many sensor pixels that are associated with each data element 352 may e.g. depend on whether binning is used or not, and equal e.g. B. In this example, the number of data elements 352 counts to N/B, where N is the total number of sensor pixels 220 (i.e. SP1 to SPN), and B=1 for no binning and B≥2 for binning. In an operation 234, the set of pixel frame data 350-t is provided to/obtained by the device 300. As indicated by the dashed box, the device 300 may e.g. perform the binning itself, in which case obtaining the final set of pixel frame data 350-t is performed as part of an internal process of the device 300 itself.

In order to combine the sets of pixel frame data 350-t (and e.g. 350-(t−1), etc.) obtained by the device 300 as part of generating an accumulation of the (repeated) sensor readouts over time, the device 300 is further configured to perform an operation 236 in which data elements 352 (or e.g. pixel lines) from different sets of pixel frame data are combined as described herein. For example, a first data element Li(t−1) found in the (first) set of pixel frame data 350-(t−1) may be combined with another, second data element L(i±D)t found in the (second) set of pixel frame data 350-t, where D is selected as explained earlier herein, depending on e.g. whether binning is used or not. As can be seen, the operation 236 includes that data elements 352 that are combined from different sets of pixel frame data are associated with different one or more (adjacent) sensor pixels 220 of the plurality of sensor pixels SP1-SPN. For example, if the data element Li(t−1) is associated with a sensor pixel SPi, the data element L(i±D)t is associated with a sensor pixel SP(i±D), i.e. a sensor pixel that is spatially offset a distance D times the first distance dy (i.e. equal to the second distance h if B=1 and K=D), and so on. While accumulating such repeated readouts over time, the device 300 builds (at least part of) an (X-ray) image 400, based on the accumulations performed so far. As each new set of pixel frame data arrives, the device 300 may add one or more new elements/lines to the image 400.

In summary of all of the above, the present disclosure provides an improved way of generating X-ray images of (moving) objects, in that it proposes to reduce the readout frequency of the detector and compensate the thus-introduced motion blur to at least some extend by increase the distance (in the scanning direction of the sensor) between pixel lines whose data are later accumulated to create a still image of a moving object. The proposed solution thus increases the efficiency of the detector, which may be particularly valuable for high-speed imaging and e.g. for multi-energy detectors where the time spent on reading out data from the sensor(s) will otherwise grow at the expense of time available for actually detecting photons/incoming radiation. The proposed solution can also be used to reduce data bandwidth in the detector from an early point of the processing chain, unlike conventional solutions in which spatial binning can reduce the bandwidth after the data has already been transferred from the sensor to e.g. a post-processing module/unit. Further, the proposed solution offers the ability to reduce the spatial resolution (by reducing the readout frequency) on-demand, while still being able to use the full potential/resolution of the sensor(s) in other situations. For example, if objects were expected to move quickly, the sensor may be replaced by a lower-resolution sensor, but then eliminating the possibility to obtain higher-resolution images for more slowly moving objects. The present disclosure and the solution envisaged herein is thus more flexible as it may still provide higher-resolution images for more slowly moving objects, by increasing the readout frequency for such objects again.

Although features and elements may be described above in particular combinations, each feature or element may be used alone without the other features and elements or in various combinations with or without other features and elements. Additionally, variations to the disclosed embodiments may be understood and effected by the skilled person in practicing the claimed disclosure as defined by the appended patent claims, from a study of the drawings, the disclosure, and the appended claims themselves. In the claims, the words “comprising” and “including” does not exclude other elements, and the indefinite article “a” or “an” does not exclude a plurality. The mere fact that certain features are recited in mutually different dependent claims does not indicate that a combination of these features cannot be used to advantage.

The following is a list of itemized exemplary embodiments in according with what has been disclosed herein:
Example 1: A device (300) for generating an X-ray image (400), wherein the device includes processing circuitry (310) configured to:—obtain, from a sensor (210) including a plurality of sensor pixels (220) spatially offset by a first distance (dy) in a scanning direction (y), a first set (350-(t−1)) of pixel frame data corresponding to a first readout time instance (t−1) and a second (350-t) set of pixel frame data corresponding to a second readout time instance (t), wherein between the first and second readout time instances is such that between the first and second readout time instances, a projection on the sensor of a point (122, 124, 126) of an imaged object (120) has had time to move a second distance (h) in the scanning direction of the sensor greater than the first distance;-combine a first data element (Li(t−1)) of the first set of pixel frame data with a second data element (L(i±D)t) of the second set of pixel frame data, wherein the first and second data elements are associated with different sensor pixels, and generate at least part of an X-ray image (400) of the object based on the combination of the first data element and the second data element.
Example 2: The device according to example 1, wherein the first data element and the second data element are each associated with one or more adjacent sensor pixels, wherein the one or more adjacent sensor pixels of the first data element are each spatially offset from one or more adjacent sensor pixels of the second data element by the second distance in the scanning direction of the sensor.
Example 3: The device according to examples 1 or 2, wherein each data element is formed from a spatial binning of readout of a first integer number (B) of adjacent sensor pixels of the plurality of sensor pixels, wherein the first integer number is equal to or larger than two (B≥2).
Example 4: The device according to any one of examples 1 to 3, wherein the second distance equals a second integer number (K) times the first distance, wherein the second integer number is equal to or larger than two (K≥2).
Example 5: The device according to examples 3 and 4, wherein the first integer number equals the second integer number (B=K).
Example 6: The device according to example 4 or 5, wherein the processing circuitry is further configured to perform the spatial binning as part of obtaining the first set and second set of pixel frame data.
Example 7: The device according to any one of examples 1 to 3, wherein each of the first and second data elements is formed from readout of a single (B=1) sensor pixel of the plurality of sensor pixels.
Example 8: The device according to any one of the preceding examples, wherein the processing circuitry is further configured to:—obtain an indication of an estimated speed of movement (|v|) of the projection on the sensor of the point of the imaged object, and adjusting, by controlling the repeated readouts of the sensor, the time difference between the first and second readout time instances based on the estimated speed of movement.
Example 9: The device according to any one of the preceding examples, wherein the processing circuitry is further configured to control a speed of movement (|v|) of the projection on the sensor of the point of the image object based on a predefined time difference between the first and second readout time instances.
Example 10: The device according to any one of the preceding examples, wherein the processing circuitry is further configured to:—implement or at least access at least one line delay element (520) configured to output a data element previously stored therein in response to receiving a new data element to be stored in the line delay element;-implement or at least access a first data structure (510) configured to store one or more data elements, and process the data elements of the first set and second set of pixel frame data one by one, including adding, for each data element, a new data element to a beginning of the first data structure, by a combination of a data element not yet stored in the first data structure to a combination of one or more data elements previously added to the first data structure, wherein one or the other of the data element not yet stored in the first data structure and the combination of one or more data elements previously added to the first data structure are first passed through the at least one line delay element.
Example 11: The device according to example 10 depending on example 4, wherein the at least one line delay element includes a number of daisy-chained line delay elements (520-1, 520-2, . . . , 520-D′) equal to at least the second integer number (D′=K).
Example 12: An X-ray detector (200), including one or more multiline X-ray sensors (210) and a device (300) according to any one of examples 1 to 11 for generating an X-ray image (400) of an object based on repeated readouts from the one or more sensors.
Example 13: The detector according to example 12, including at least two sensors and wherein the device is configured for processing data readout from each sensor in parallel to create the X-ray image.
Example 14: The detector according to example 12 or 13, wherein the detector is a photon- counting detector.
Example 15: An X-ray imaging system (100) for generating an X-ray image, including a multiline X-ray detector (200) according to any one of examples 12 to 14, or one or more multiline X-ray sensors (210) and a device (300) according to any one of examples 1 to 11.
Example 16: The system according to example 15, further including at least one X-ray source (110) configured to radiate X-rays towards the one or more multiline X-ray sensors.
Example 17: The system according to example 15 or 16, further including a motion apparatus (130) for moving an object (120) to be imaged relative the detector, wherein the device is further configured to control a relative speed of movement between the object and one or more sensors (201) of the detector.
Example 18: A method (700) for generating an X-ray image, including:-obtaining (S710), from a sensor including a plurality of sensor pixels spatially offset by a first distance (dy) in a scanning direction (y), a first set of pixel frame data corresponding to a first readout time instance and a second set of pixel frame data corresponding to a second readout time instance, wherein between the first and second readout time instances, a projection on the sensor of a point of an imaged object has had time to move a second distance in the scanning direction of the sensor greater than the first distance;-combining a first data element of the first set of pixel frame data with a second data element of the second set of pixel frame data, wherein the first and second data elements are associated with different sensor pixels, and generating (S730) at least part of an X-ray image of the object based on the combination of the first data element and the second data element.

1. A device for generating an X-ray image, wherein the device comprises processing circuitry configured to:
obtain, from a sensor comprising a plurality of sensor pixels spatially offset by a first distance in a scanning direction, a first set of pixel frame data corresponding to a first readout time instance and a second set of pixel frame data corresponding to a second readout time instance, wherein a time difference between the first readout time instance and second readout time instance is such that a projection on the sensor of a point of an imaged object has had time to move a second distance in the scanning direction of the sensor greater than the first distance;
combine a first data element of the first set of pixel frame data with a second data element of the second set of pixel frame data, wherein the first data element and the second data element are associated with different sensor pixels; and
generate at least part of an X-ray image of the object based on the combination of the first data element and the second data element.

2. The device according to claim 1, wherein the first data element and the second data element are each associated with one or more adjacent sensor pixels, wherein the one or more adjacent sensor pixels of the first data element are each spatially offset from one or more adjacent sensor pixels of the second data element by the second distance in the scanning direction of the sensor.

3. The device according to claim 1, wherein each data element is formed from a spatial binning of readout of a first integer number of adjacent sensor pixels of the plurality of sensor pixels, wherein the first integer number is equal to or larger than two.

4. The device according to claim 3, wherein the second distance equals a second integer number times the first distance, wherein the second integer number is equal to or larger than two.

5. The device according to claim 4, wherein the first integer number equals the second integer number.

6. The device according to claim 3, wherein the processing circuitry is further configured to perform the spatial binning as part of obtaining the first set of pixel frame data and the second set of pixel frame data.

7. The device according to claim 1, wherein each of the first data element and the second data element is formed from readout of a single sensor pixel of the plurality of sensor pixels.

8. The device according to claim 1, wherein the processing circuitry is further configured to:
obtain an indication of an estimated speed of movement of the projection on the sensor of the point of the imaged object; and
adjust, by controlling repeated readouts of the sensor, the time difference between the first readout time instance and the second readout time instance based on the estimated speed of movement.

9. The device according to claim 1, wherein the processing circuitry is further configured to control a speed of movement of the projection on the sensor of the point of the imaged object based on a predefined time difference between the first and second readout time instances.

10. The device according to claim 1, wherein the processing circuitry is further configured to:
implement or access at least one line delay element configured to output a data element previously stored therein in response to receiving a new data element to be stored in the line delay element;
implement or access a first data structure configured to store one or more data elements; and
process the data elements of the first set and second set of pixel frame data one by one, comprising adding, for each data element, a new data element to a beginning of the first data structure, by a combination of a data element not yet stored in the first data structure to a combination of one or more data elements previously added to the first data structure, wherein one or the other of the data element not yet stored in the first data structure and the combination of one or more data elements previously added to the first data structure are first passed through the at least one line delay element.

11. The device according to claim 10, wherein:
the second distance equals a first integer number times the first distance;
the first integer number is equal to or larger than two; and
the at least one line delay element comprises a number of daisy-chained line delay elements equal to the first integer number.

12. The device according to claim 1, further comprising an X-ray detector coupled to the processing circuitry, the X-ray detector comprising one or more multiline X-ray sensors.

13. The device according to claim 12, wherein:
the X-ray detector comprises at least two multiline X-ray sensors; and
the processing circuitry is configured for processing data readout from each of the multiline X-ray sensors in parallel to create the X-ray image.

14. The device according to claim 12, wherein the X-ray detector comprises a photon- counting detector.

15. An X-ray imaging system for generating an X-ray image, the X-ray imaging system comprising:
a multiline X-ray detector or one or more multiline X-ray sensors; and
the device according to claim 1.

16. The X-ray imaging system according to claim 15, further comprising at least one X-ray source configured to radiate X-rays towards the multiline X-ray detector or the one or more multiline X-ray sensors.

17. The X-ray imaging system according to claim 15, further comprising a motion apparatus for moving an object to be imaged relative to the multiline X-ray detector or the one or more multiline X-ray sensors, wherein the processing circuitry is further configured to control a relative speed of movement between the object and the multiline X-ray detector or the one or more multiline X-ray sensors.

18. A method for generating an X-ray image, the method comprising:
obtaining, from a sensor comprising a plurality of sensor pixels spatially offset by a first distance in a scanning direction, a first set of pixel frame data corresponding to a first readout time instance and a second set of pixel frame data corresponding to a second readout time instance, wherein between the first and second readout time instances, a projection on the sensor of a point of an imaged object has had time to move a second distance in the scanning direction of the sensor greater than the first distance;
combining a first data element of the first set of pixel frame data with a second data element of the second set of pixel frame data, wherein the first data element and the second data element are associated with different sensor pixels, and
generating at least part of an X-ray image of the object based on the combination of the first data element and the second data element.
